(19) United States
(12) Patent Application Publication
(10) Pub. No.: US 2020/0049700 A1
(43) Pub. Date: Feb. 13, 2020

(54) NANOPARTICLE-ENHANCED ENZYMATIC IMMUNE BIOSENSOR ASSAY FOR ANTIGEN DETECTION

(71) Applicant: University of South Florida, Tampa, FL (US)

(72) Inventors: Shyam Mohapatra, Lutz, FL (US); Subhra Mohapatra, Lutz, FL (US)

(21) Appl. No.: 16/608,056

(22) PCT Filed: Apr. 26, 2018
(86) PCT No.: PCT/US2018/029593

§ 371 Date: Oct. 24, 2019

Related U.S. Application Data
(60) Provisional application No. 62/618,896, filed on Jan. 18, 2018.
(60) Provisional application No. 62/490,436, filed on Apr. 26, 2017.

(51) Int. Cl. G01N 33/543; G01N 33/574

(52) U.S. Cl. CPC G01N 33/54346; G01N 33/57415; G01N 2333/904; G01N 2333/928; G01N 2800/347

(57) ABSTRACT

Provided are methods of detecting antigens in a sample by methods using a nanoparticle-enhanced enzymatic immune bio sensor. Methods of detecting abnormal kidney function in a subject and methods of detecting cancer in a subject are provided. Also provided are kits for detecting antigens.

### CROSS-REFERENCE TO RELATED APPLICATION(S)

This application claims priority to U.S. Provisional Patent Application No. 62/490,436, filed on Apr. 26, 2017 and U.S. Provisional Patent Application No. 62/618,896, filed on Jan. 18, 2018, the entire contents of each of which are fully incorporated herein by reference.

### STATEMENT OF GOVERNMENT SUPPORT

This invention was made with government support under Grant Number NIH RO1 CA152005 awarded by the National Institute of Health and Grant Numbers IK6 BX003778-02, IK6BX004212, and BX003685 awarded by the Veteran's Administration. The government has certain rights in the invention.

### FIELD

Provided are methods of using nanoparticle-enhanced enzymatic immune biosensor assays to detect antigens.

### INTRODUCTION

Biomarkers are any measurable characteristics of an organism that reflect a particular physiological state. Biomarkers may be compounds isolated from a sample and used as an indicator of the presence or severity of a particular disease state. Biomarkers can also be used to assess the effectiveness of particular therapies in ameliorating the effects of a disease. Assaying biomarkers to monitor a patient's reaction to a particular drug, may determine whether treatment is effective for that individual by measuring drug response rate or toxic effects associated with the drug. Biomarkers may also serve to detect disease at an early stage, thus increasing survival rates.

### SUMMARY

In one aspect, provided are methods of detecting an antigen in a subject. The method may include method of detecting an antigen in a subject, the method comprising: a) contacting a sample from the subject with i) a first antibody conjugated to a first nanoparticle; ii) a second antibody conjugated to a second nanoparticle, wherein the nanoparticle is conjugated to an enzyme, wherein the first and second antibodies bind an antigen; and iii) a substrate capable of being converted to a product by the enzyme; b) determining the level of the product; c) comparing the level of product to a reference level of product; thereby detecting the level of antigen.

In another aspect, provided are kits for detecting the level of an antigen in a subject. The kit may comprise: a first antibody conjugated to a first nanoparticle; a second antibody conjugated to a second nanoparticle, wherein the nanoparticle is conjugated to an enzyme, wherein the first and second antibodies bind an antigen; and a substrate capable of being converted to a product by the enzyme.

In one aspect, provided are methods of detecting abnormal kidney function. The method may include a) obtaining a sample from the subject; b) contacting the sample with: i) a first antibody conjugated to a first nanoparticle; ii) a second antibody conjugated to a second nanoparticle, wherein the nanoparticle is conjugated to an enzyme, wherein the first and second antibodies bind an antigen; and iii) a substrate capable of being converted to a product by the enzyme c) determining the level of the product; d) comparing the level of product to a reference level of product; and e) identifying the subject as having abnormal kidney function when the level of the product is different than the level of product from a reference level of product.

In another aspect, provided are methods for detecting cancer in a subject. The method may include: a) obtaining a sample from the subject; b) contacting the sample with: i) a first antibody conjugated to a first nanoparticle; ii) a second antibody conjugated to a second nanoparticle, wherein the nanoparticle is conjugated to an enzyme, wherein the first and second antibodies bind an antigen; and iii) a substrate capable of being converted to a product by the enzyme; c) determining the level of the product; d) comparing the level of product to a reference level of product; and e) identifying the subject as having cancer when the level of the product is different than the level of product from a reference level of product.

In another aspect, provided is a method for detecting an antigen in a subject. The method may comprise: a) obtaining a sample from a subject; b) contacting a sample from the subject with: i) an antigen specific antibody conjugated to a nanoparticle; ii) an antigen conjugated to an enzyme; and iii) a substrate capable of being converted to a product by the enzyme; b) determining the level of product; and c) comparing the level of product to a reference level of product; thereby detecting the level of biomarker.

In another aspect, provided are kits for detecting the level of an antigen in a subject. The kit may comprise: a) an antigen specific antibody conjugated to a nanoparticle; b) an antigen conjugated to an enzyme; and c) a substrate capable of being converted to a product by the enzyme.

In another aspect, described are methods for detecting abnormal kidney function. The methods may comprise: a) obtaining a sample from the subject; b) contacting the sample with: i) an antigen specific antibody conjugated to a nanoparticle; an antigen conjugated to an enzyme; and ii) a substrate capable of being converted to a product by the enzyme; b) determining the level of product; comparing the level of product to a reference level of product; and c) identifying the subject as having abnormal kidney function when the level of product is different than the level of product from a reference level of product.

In another aspect, described are methods for detecting cancer. The methods may comprise: a) obtaining a sample from the subject; b) contacting the sample with: i) an antigen specific antibody conjugated to a nanoparticle; ii) an antigen conjugated to an enzyme; and iii) a substrate capable of being converted to a product by the enzyme; c) determining the level of product; d) comparing the level of product to a reference level of product; and e) identifying the subject as having cancer when the level of product is different than the level of product from a reference level of product.

Other aspects of the invention will become apparent by consideration of the detailed description and accompanying drawings.

### BRIEF DESCRIPTION OF THE DRAWINGS

FIG. 1 is a schematic of quantifying albumin by a dual nanoparticle-enhanced enzymatic immune biosensor (DNEI) assay that uses a glucose meter for quantitative expression of disease antigens.

FIG. 2 is a schematic of quantifying creatinine by a dual nanoparticle-enhanced enzymatic immune biosensor (DNEI) assay that uses a glucose meter for quantitative expression of disease antigens.

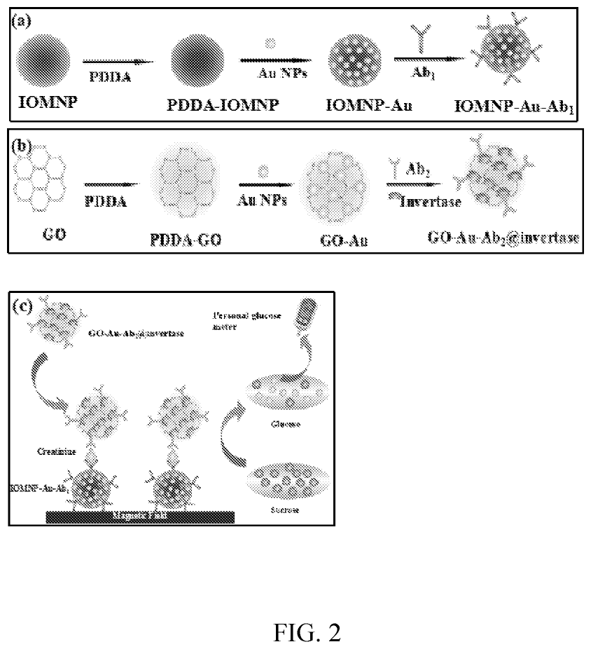

FIG. 3 depicts a sensitive point-of-detection of cortisol by personal glucose meter.

FIG. 4 depicts a sensitive point-of-detection of cortisol by personal glucose meter.

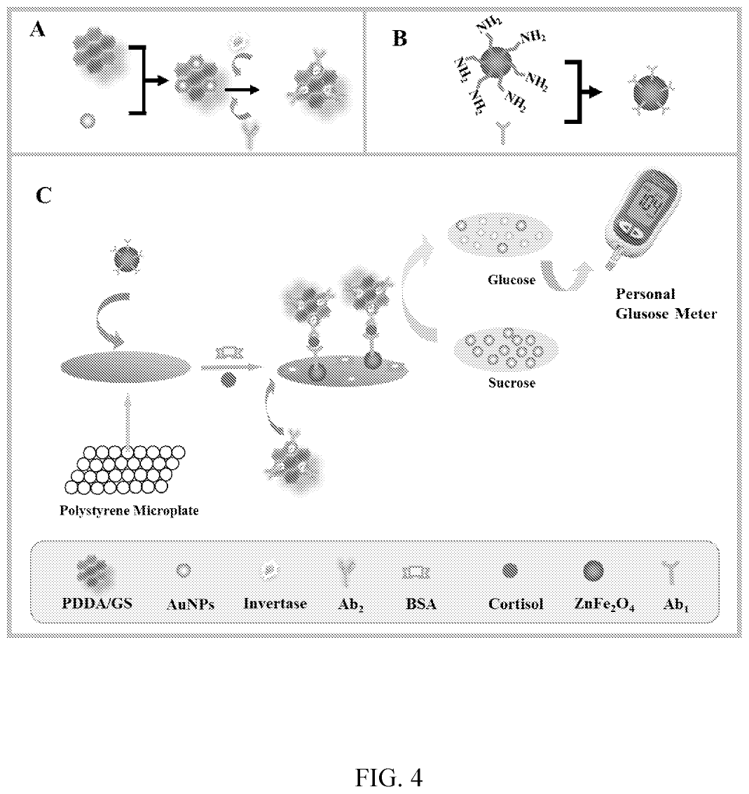

FIG. 5 depicts cortisol.

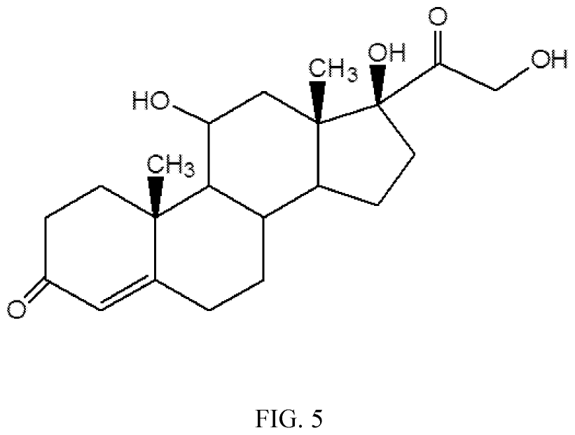

FIG. 6 is a schematic showing a competitive immunoassay using PDDA-AuNPs.

FIG. 7 shows cytric functionalized magnetic particles.

FIG. 8 shows the preparation of gold-polyelectrolyte nanocomposites was a simple, single step approach, where PDDA acts as a reducing and stabilizing agent.

FIG. 9 shows the morphology of fabricated Fe₃O₄—Au nanocomposite was studied by TEM.

FIG. 10 shows the morphology of fabricated Fe₃O₄—Au-bioconjugate was studied by TEM.

FIG. 11 depicts TEM images of (A) Fe₃O₄ Nanoparticles; (B) Fe₃O₄—Au nanoparticles; (C) Graphene oxide (GO); (D) GO-Au. XRD patterns of (E) of Fe₃O₄—Au nanocomposites and EDS of GO-Au. (G) Electrochemical impedance spectra of a) bare GCE; (b) Fe₃O₄/GCE; (c) Antibody 1 (Ab1) conjugated Fe₃O₄/GCE; (d) BSA/Ab1-Fe₃O₄/GCE; (e) Albumin/Ab1-Fe₃O₄/GCE; (f) GO-Au-Ab2 with invertase/Albumin/Ab1-Fe₃O₄/GC in 0.2 mol/L KCl containing 2.5 mmol/L K3Fe(CN)6. (H) Calibration curve of the immunosensor toward different concentrations of albumin. (I) Demonstration of feasibility of measuring HER2 antigen levels by using DNEI assay. The dual nanoparticle system comprised of citric acid-functionalized magnetic nanoparticles and fluorinated graphene-silver oxide NPs. The lowest detection limit for the glucometer coincided with 22 ng/ml of HER2 indicating 10-fold higher sensitivity of HER2 detection than the commercial sandwich ELISA.

FIG. 12 is a schematic showing the sample enrich process, trace tag building process, and silver enhancement detecting.

FIG. 13 shows(a) TEM of IOMNP-Au-Ab1 complex (b) AuNP PSD as per TEM (c) AuNP PSD as per light scattering (d) IOMNP PSD as per TEM (e) IOMNP PSD as per light scattering.

FIG. 14 is a diagram showing invertase and cortisol binding to nanoparticles.

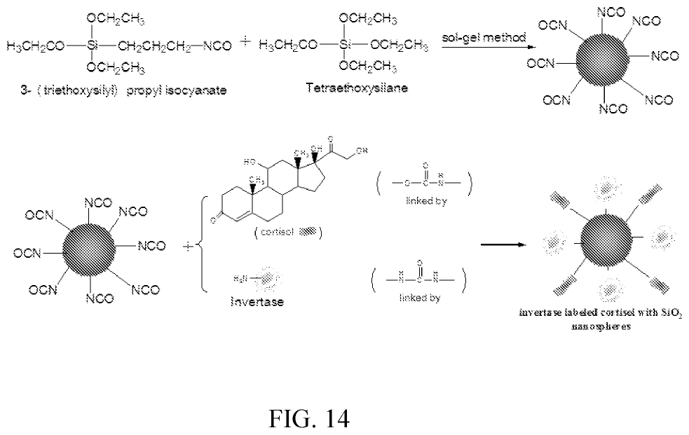

FIG. 15 shows (a) TEM of second complex and (b) GO PSD as per light scattering.

FIG. 16 shows the results of testing the performance of the creatinine system. The creatinine concentration was varied while keeping all other conditions constant. Each concentration was sampled twice and the PGM readings were tabulated (FIG. 16A) and the average reading for each concentration was plotted to formed a standard calibration curve shown in FIG. 16B. The second calibration curve (FIG. 4C) was formed for the region of interest (0.2 mg/dL-1.4 mg/dL). The curve shows a linear regression, with low standard deviation values. Finally, albumin and did not yield a PGM reading thus confirming the specificity of the assay to creatinine.

FIG. 17 is a graph showing the calibration curve for standardizing sample creatinine concentration to PGM reading.

FIG. 18 shows the results from creatinine.

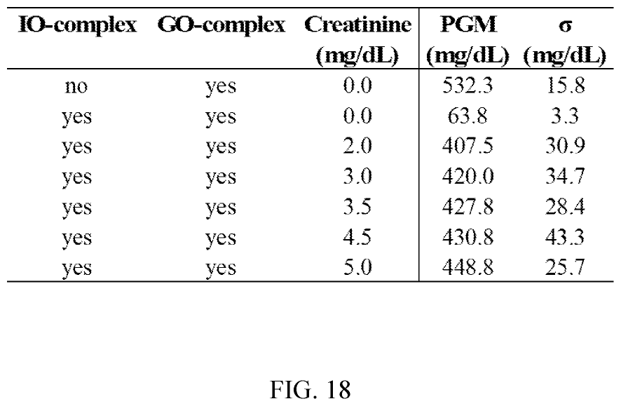

FIG. 19 is a schematic that shows ultrasensitive electrochemical immunosensing detection of cortisol.

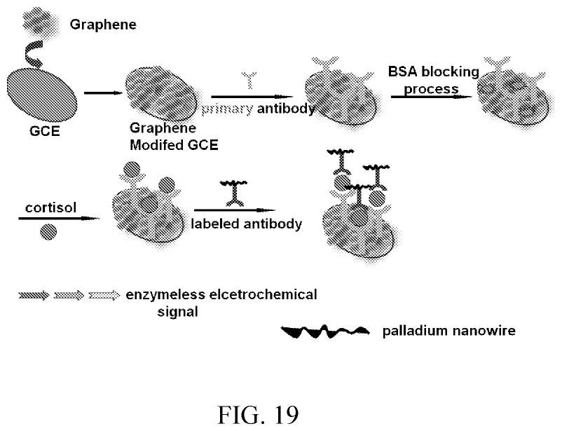

FIG. 20 is a schematic that shows the sensitive detection of cortisol by electrochemical method combining molecular imprinting technique.

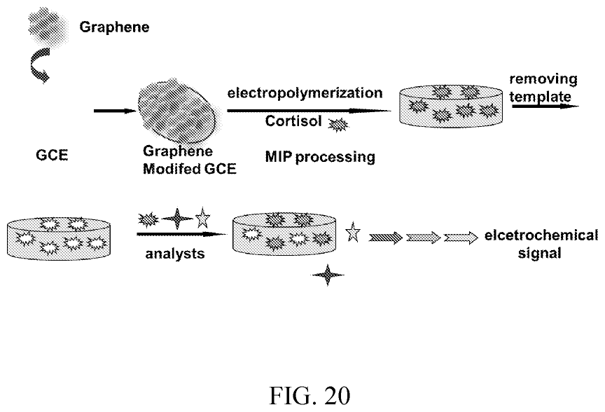

FIG. 21 is a schematic that shows imprinting.

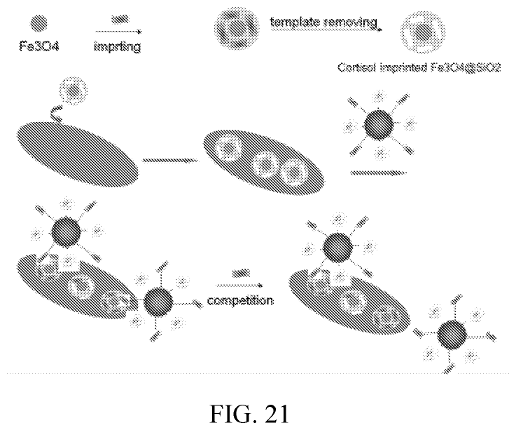

Before any embodiments of the invention are explained in detail, it is to be understood that the invention is not limited in its application to the details of construction and the arrangement of components set forth in the following description or illustrated in the accompanying drawings. The invention is capable of other embodiments and of being practiced or of being carried out in various ways.

### DETAILED DESCRIPTION

Disclosed herein are methods for the detection of an antigen in a sample. The method may include the use of a dual nanoparticle-enhanced enzymatic immune biosensor (DNEI) assay. The method may include the use of a single nanoparticle-enhanced enzymatic immune biosensor assay. The herein described methods can quantitatively detect antigens. The antigen may be a biomarker. The biomarker may be a metabolite. Also disclosed are methods for detecting abnormal kidney function and methods for detecting cancer.

### 1. Definitions

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The terms “administration” or “administering” as used herein may include the process in which the compounds or compositions as described herein, alone or in combination with other compounds or compositions, are delivered to a subject. The compound or compositions may be administered in various routes including, but not limited to, oral, parenteral (including intravenous, intra-arterial, and other appropriate parenteral routes), intrathecally, intramuscularly, subcutaneously, colonically, rectally, and nasally, transcutaneously, among others. The dosing of the agents, compounds, and compositions described herein to obtain a therapeutic or prophylactic effect may be determined by the circumstances of the subject, as known in the art. The dosing of a subject herein may be accomplished through individual or unit doses of the compounds or compositions herein or by a combined or prepackaged or pre-formulated dose of a compounds or compositions.

Administration may depend upon the amount of compound or composition administered, the number of doses, and duration of treatment. For example, multiple doses of the agent may be administered. The frequency of administration of the compound or composition may vary depending on any of a variety of factors. The duration of administration of the compound or composition, e.g., the period of time over which the compound or composition is administered, may vary, depending on any of a variety of factors, including subject response, etc.

The amount of the compound or composition contacted (e.g., administered) may vary according to factors such as the degree of susceptibility of the individual, the age, sex, and weight of the individual, idiosyncratic responses of the individual, the dosimetry, and the like. Detectably effective amounts of the compounds or compositions of the present disclosure may also vary.

As used in this specification and the appended claims, the singular forms “a”, “an”, and “the” include plural referents unless the content clearly dictates otherwise. As used in this specification and the appended claims, the term “or” is generally employed in its sense including “and/or” unless the context clearly dictates otherwise.

As used herein, the terms “cancer cells,” “neoplastic cells,” “neoplasia,” “tumor,” and “tumor cells” (used interchangeably) refer to cells which exhibit relatively autonomous growth so that they exhibit an aberrant growth phenotype characterized by a significant loss of control of cell proliferation (i.e., de-regulated cell division). These cells may be malignant or benign.

The terms “cell,” “cell line,” and “cell culture” include progeny. It is also understood that all progeny may not be precisely identical in DNA content due to deliberate or inadvertent mutations. Variant progeny that have the same function or biological property, as screened for in the originally transformed cell, are included.

The terms “chemotherapeutic agent” and “anti-cancer drug” and “drug for the treatment of cancer” and as used herein, may be used interchangeably.

The term “control” or “normal control”, as used herein, refers to an alternative subject or sample used in an experiment for comparison purpose and included to minimize or distinguish the effect of variables other than an independent variable. A control may be a healthy subject (e.g., a subject with normal kidney function or a subject without cancer). A control may be a non-cancerous cell line. A control may be a sample taken from a healthy subject. A control sample may be from a subject that does not have cancer. A control sample may be from a subject that does not have abnormal kidney function.

The use of “including,” “comprising,” or “having” and variations thereof herein is meant to encompass the items listed thereafter and equivalents thereof as well as additional items. Any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

The terms “label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromagens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

A “pharmaceutically acceptable excipient,” “pharmaceutically acceptable diluent,” “pharmaceutically acceptable carrier,” or “pharmaceutically acceptable adjuvant” means an excipient, diluent, carrier, and/or adjuvant that are useful in preparing a pharmaceutical composition that are generally safe, non-toxic and neither biologically nor otherwise undesirable, and includes an excipient, diluent, carrier, and adjuvant that are acceptable for veterinary use and/or human pharmaceutical use. “A pharmaceutically acceptable excipient, diluent, carrier and/or adjuvant” as used herein includes one or more such excipients, diluents, carriers, and adjuvants.

The terms “reference” or “reference level”, as used herein, may be a values(s) for a physiologic measurement. The measurement may be from a healthy subject. For example the healthy subject may not have a disease. The healthy subject may have normal kidney function. The healthy subject may not have cancer. The measurement may be from an unhealthy subject. For example the unhealthy subject may have a disease. The unhealthy subject may have kidney disease. The unhealthy subject may have cancer. The unhealthy subject may be suspected of having a disease or condition (e.g., abnormal kidney function, cancer). The reference level may be a basis for comparison to a measurement from a subject that has or may be suspected of having a disease. The reference level may be of a product. The reference level may be of a biomarker. The reference level may be of a metabolite. The reference level may be generated from a standard curve. The reference level may be determined from a sample obtained from a healthy subject. The reference level may be a cutoff value.

The term “sample” or “biological sample” or “test sample” as used herein may mean any sample in which the presence and/or level of a substrate is to be detected or determined. Samples may include a medical sample. Samples may include any biological fluid or tissue, including, but not limited to blood, whole blood, fractions of blood such as plasma and serum, urine, cerebrospinal fluid, amniotic fluid, saliva, sweat, a tumor cell lysate, a pre-surgical biopsy, a biopsy obtained during surgery, a post-surgical biopsy, muscle, interstitial fluid, sweat, saliva, tears, synovial fluid, bone marrow, cerebrospinal fluid, nasal secretions, sputum, amniotic fluid, bronchoalveolar lavage fluid, lung tissue, peripheral blood mononuclear cells (PBMC), total white blood cells, monocytes, lymph node cells, spleen cells, tonsil cells, skin, or combinations thereof. In some embodiments, the sample comprises a biological tissue. In some embodiments the sample comprises a cell lysate. In some embodiments, the sample comprises a tumor cell lysate, a pre-surgical biopsy, a biopsy obtained during surgery, or a post-surgical biopsy. Samples can be obtained by any means known in the art. The sample can be used directly as obtained from a subject or can be pre-treated, such as by filtration, distillation, extraction, concentration, centrifugation, inactivation of interfering components, addition of reagents, and the like, to modify the character of the sample in some manner as discussed herein or otherwise as is known in the art. In some embodiments, the sample may be blood, serum, urine, cerebrospinal fluid, amniotic fluid, saliva, or sweat.

As used herein, the term “subject,” “patient,” or “organism” includes humans and mammals (e.g., mice, rats, pigs, cats, dogs, and horses). Typical subjects to which an agent(s) of the present disclosure may be administered may include mammals, particularly primates, especially humans. For veterinary applications, suitable subjects may include, for example, livestock such as cattle, sheep, goats, cows, swine, and the like; poultry such as chickens, ducks, geese, turkeys, and the like; and domesticated animals particularly pets such as dogs and cats. For diagnostic or research applications, suitable subjects may include mammals, such as rodents (e.g., mice, rats, hamsters), rabbits, primates, and swine such as inbred pigs and the like. The subject may have abnormal kidney function. The subject may have cancer. The subject may have breast cancer.

The “therapeutically effective amount” for purposes herein may be determined by such considerations as are known in the art. A therapeutically effective amount of a compound may include the amount necessary to provide a therapeutically effective result in vivo. The amount of the compound or composition must be effective to achieve a response, including but not limited to a total prevention of (e.g., protection against) of a condition, improved survival rate or more rapid recovery, improvement or elimination of symptoms associated with the condition (such as abnormal kidney function or cancer), or other indicators as are selected as appropriate measures by those skilled in the art. As used herein, a suitable single dose size includes a dose that is capable of preventing or alleviating (reducing or eliminating) a symptom in a subject when administered one or more times over a suitable time period. The “therapeutically effective amount” of a compound or composition as described herein may depend on the route of administration, type of subject being treated, and the physical characteristics of the subject. These factors and their relationship to dose are well known to one of skill in the medicinal art, unless otherwise indicated.

As used herein, “treat”, “treatment”, “treating”, and the like refer to acting upon a condition with an agent to affect the condition by improving or altering it. The condition includes, but is not limited to infection, such as those caused by abnormal kidney function or cancer. The aforementioned terms cover one or more treatments of a condition in a subject (e.g., a mammal, typically a human or non-human animal of veterinary interest), and include: (a) reducing the risk of occurrence of the condition in a subject determined to be predisposed to the condition but not yet diagnosed, (b) impeding the development of the condition, and/or (c) relieving the condition, e.g., causing regression of the condition and/or relieving one or more condition symptoms (e.g., treating abnormal kidney function or cancer).

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Method of Detecting an Antigen

Provided herein are methods of detecting an antigen in a sample. The methods may detect one or more antigens. The method may include the use of a dual nanoparticle-enhanced enzymatic immune biosensor assay. The method may include the use of a single nanoparticle-enhanced enzymatic immune biosensor assay. A first antibody may be specific to an antigen. A first antibody and second antibody may be specific to the same antigen. The method may then include determining the level of the product and comparing the level of product to a reference level of product, thereby detecting the level of the antigen. The antigen may be a biomarker. The biomarker may be a kidney function biomarker. The biomarker may be a cancer biomarker. The biomarker may be a metabolite.

### a. Dual Nanoparticle-Enhanced Enzymatic Immune Biosensor Assay

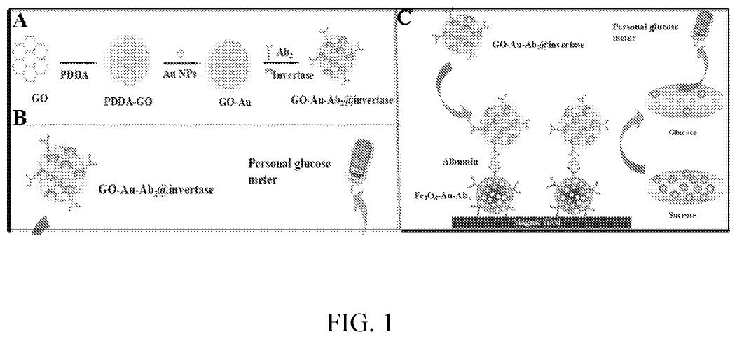

The dual nanoparticle-enhanced biosensor assay relies on two populations of nanoparticles. Each population of nanoparticles is conjugated to an antibody that binds to a specific antigen of interest. The first antibody and second antibody may be the same antibody. The first antibody and second antibody may be different, but bind to the same antigen. The first antibody and second antibody may bind to the same antigen, but bind a different epitope on the antigen. The second population of nanoparticles may be bound to an enzyme (e.g., invertase). The sample may be subjected to an external magnetic field, to retain the nanoparticles in the sample. The sample may be washed while subjected to the magnetic field. The nanoparticles remain in the sample, and are bound by the conjugated antibody, to sample antigen. A substrate is added to the sample. The enzyme bound to the nanoparticle will convert the substrate to a product. Addition of a substrate (e.g., sucrose) to the reaction vessel produces a product (e.g., glucose) that is directly proportional to the sample antigen present. A schematic of the dual nanoparticle enhanced biosensor assay is shown in FIG. 1. The antigen may be a biomarker. The biomarker may be a kidney function biomarker. The biomarker may be a cancer biomarker. The biomarker may be a metabolite. The metabolite may be associated with kidney function. The metabolite may be associated with cancer.

### b. Single Nanoparticle-Enhanced Enzymatic Immune Biosensor Assay

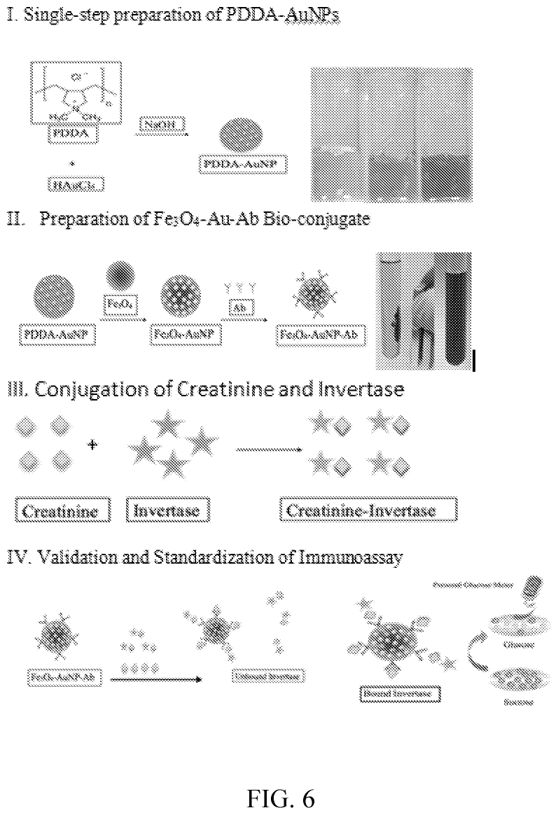

The single nanoparticle-enhanced biosensor assay relies on one population of nanoparticles. The nanoparticles are conjugated to antibodies that bind to an antigen of interest. An enzyme (e.g., invertase) is conjugated to an exogenously supplied specific antigen of interest. The exogenously supplied specific antigen of interest may be a control. The quantity of the enzyme-bound control antigen may be known. A sample, which may or may not contain the antigen of interest, is contacted with a) the nanoparticle conjugated to an antibody, and b) the enzyme-bound control antigen. The enzyme-bound control antigen competes with the sample antigen for binding to the nanoparticle-conjugated antibody. The sample may be subjected to an external magnetic field, to retain the nanoparticles in the sample. The sample may be washed while subjected to the magnetic field. The nanoparticles remain in the sample, and are bound by the conjugated antibody, to sample antigen and/or control antigen. A substrate is added to the sample. The enzyme bound to the control antigen will convert the substrate to a product. Addition of a substrate (e.g., sucrose) to the reaction vessel produces a product (e.g., glucose) that is inversely proportional to the sample antigen present. This value may be calculated based on a standard curve generated with known concentrations of the control antigen. A schematic of the single nanoparticle-enhanced enzymatic immune biosensor assay is shown in FIG. 6. The antigen may be a biomarker. The biomarker may be a kidney function biomarker. The biomarker may be a cancer biomarker. The biomarker may be a metabolite. The metabolite may be associated with kidney function. The metabolite may be associated with cancer.

### c. Antibodies

Examples of antibodies that can be used with the methods disclosed herein include, but are not limited to a polyclonal antibody, a monoclonal antibody, a human antibody, an immunoglobulin molecule, a disulfide linked Fv, a monoclonal antibody, an affinity matured, a scFv, a chimeric antibody, a single domain antibody, a CDR-grafted antibody, a diabody, a humanized antibody, a multi-specific antibody, a Fab, a dual specific antibody, a DVD, a Fab′, a bispecific antibody, a F(ab′)2, a Fv, and combinations thereof. The antibody may bind to an antigen. The antibody may bind to a biomarker. The antibody may bind specific to a kidney function biomarker. The antibody may be an anti-creatinine antibody. The antibody may bind to a cancer biomarker. The antibody may be an anti-HER2 antibody. The antibody may be an anti-ER antibody. The antibody may bind to a metabolite. The metabolite may be a kidney function metabolite. The metabolite may be a cancer metabolite. The first antibody and second antibody may be the same antibody. The first antibody and second antibody may be different, but bind to the same antigen. The first antibody and second antibody may bind to the same antigen at different locations on the antigen. The first antibody and the second antibody may bind to a same epitope of the antigen. The first antibody and the second antibody may bind to a different epitope of the antigen. The first and second antibody may have specificity for different epitope(s) of the same antigen. The first antibody may be an antibody raised in an animal species different from that of the first antibody. Immobilized antibodies or fragments thereof may be incorporated into the methods. The antibody may be immobilized onto a variety of supports, such as magnetic or chromatographic matrix particles, the surface of an assay plate (such as microtiter wells), pieces of a solid substrate material, and the like. The antibody may be conjugated to a nanoparticle. The antibody may be labeled. The antibody may be conjugated to a detectable label. In some embodiments, a first antibody may be conjugated to a first nanoparticle. A second antibody may be conjugated to a second nanoparticle. A second antibody may be conjugated to a second nanoparticle. A second antibody and an enzyme may be conjugated a second nanoparticle. Antibodies may be bound to nanoparticles by covalent binding through the reaction between the amino groups in antibody with carboxylic groups in fluorinated graphene oxide.

### d. Nanoparticles

Nanoparticles may be particles between 1 and 100 nanometers in size. Methods for generating nanoparticles include, but are not limited to gas condensation, attrition, chemical precipitation, pyrolysis and hydrothermal synthesis. In attrition, macro- or micro-scale particles may be ground in a ball mill, a planetary ball mill, or other size-reducing mechanism. The surface coating of nanoparticles may determine their properties, including stability, solubility, and targeting. The nanoparticles may have magnetic properties. The nanoparticles may be immobilized onto a variety of supports, such as magnetic or chromatographic matrix particles, the surface of an assay plate (such as microtiter wells), pieces of a solid substrate material, and the like. The nanoparticles may be one or more of Fe₃O₄ nanoparticle or a graphene oxide nanoparticle. The nanoparticles may be functionalized. The nanoparticle may be functionalized with thiolated gold. The nanoparticle may be conjugated to an antibody. The nanoparticle may be conjugated to an enzyme. Antibodies may be bound to nanoparticles by covalent binding through the reaction between the amino groups in antibody with carboxylic groups in fluorinated graphene oxide.

There may be one population of nanoparticles used in the methods disclosed herein. There may be multiple populations of nanoparticles used in the methods disclosed herein. In some embodiments there may be two different populations of nanoparticles. The first nanoparticle may be a Fe₃O₄ nanoparticle functionalized with thiolated gold. The second nanoparticle may be a graphene oxide nanoparticle conjugated to thiolated gold. The first nanoparticle may be a citric acid functionalized Fe₃O₄ aqueous colloidal magnetic nanoparticle. The second nanoparticle may be a fluorinated graphene oxide-silver nanoparticle. The nanoparticle may be conjugated to an antibody. The nanoparticle may be conjugated to multiple antibodies. The nanoparticle may be conjugated to an enzyme.

### e. Detection

The method may include determining the level of the product and comparing the level of product to a reference level of product, thereby detecting the level of the antigen. The substrate may be converted to a product. The product may be converted from a substrate. The product may be converted by an enzyme. In some embodiments, the substrate may be, but is not limited to, sucrose or acetoacetic acid. In some embodiments the enzyme may be, but is not limited to, invertase or βHB-hydrogenase. In some embodiments, the product may be, but is not limited to, glucose, βhydroxybutyrate, or a ketone. In some embodiments, the substrate may be sucrose, the enzyme may be invertase, and the product may be glucose. In some embodiments, the substrate may be acetoacetic acid the enzyme may be βHB-hydrogenase, and the product may be βhydroxybutyrate.

The level of the product from the subject's sample may be compared to a standard curve. The level of product from a subject's sample may be compared to the level of product from a normal control sample. For example a normal control sample may be a sample from a healthy individual. The level of product from a subject's sample may be compared to the level of product from an abnormal control sample. For example, an abnormal sample may be from a subject with abnormal kidney function or a subject with cancer. In some embodiments, the methods may include comparing the level of product to a reference level of product. The reference level of product may correspond to a level of antigen, biomarker, or metabolite, for example. Comparing the level of product to a reference level of product may thereby detect the level of an antigen.

In some embodiments, the reference level of the antigen is the level of the antigen in a control sample. In some embodiments, the reference level of the antigen is a cutoff level. In some embodiments, the cutoff value is determined by a receiver operating curve (ROC) analysis from biological samples of a patient group. In some embodiments, the cutoff level is determined by a mean plus 2 standard deviation analysis of multiple control samples. In some embodiments, the subject has kidney disease. In some embodiments, the methods further indicate administering pharmaceutical agents to the subject. In some embodiments, the subject has cancer. In some embodiments, the methods may further comprise administering one or more pharmaceutical agents to the subject. In some embodiments, the methods may further comprise administering one or more a chemotherapeutic agents to the subject. In some embodiments, the methods may further comprise administering hormone therapy to the subject.

### f. Biomarker

An antigen may be detected by the methods disclosed herein. The antigen may be a biomarker. A Biomarker may be detected by the methods disclosed herein. A biomarker may be a substance or process that is indicative of a biological state or condition. A biomarker may indicate normal kidney function in a subject. A biomarker may indicate abnormal kidney function in a subject. A biomarker may indicate the presence of disease in the body. A biomarker may indicate cancer in subject. Biomarkers include, but are not limited to metabolic, genetic, epigenetic, proteomic, glycomic, and imaging biomarkers. Biomarkers may be used for disease detection, diagnosis, prognosis, stage monitoring, indicating the treatment that may be the most effective, monitoring the effectiveness of treatment, and epidemiology. Biomarkers may be the target of the first and second antibodies of the methods disclosed herein. Biomarkers may be assayed from samples from a patient. Biomarkers may be assayed from body fluids. Biomarkers may be assayed from blood, serum, urine, cerebrospinal fluid, amniotic fluid, saliva, sweat, a tumor cell lysate, a pre-surgical biopsy, a biopsy obtained during surgery, and/or a post-surgical biopsy. In some embodiments the biomarker is a metabolite. In some embodiments, the biomarker is a kidney function biomarker. In some embodiments, the biomarker is a cancer biomarker.

### i. Kidney Function Biomarker

The methods disclosed herein may detect a kidney function. The methods disclosed herein may detect a kidney function antigen. The methods disclosed herein may detect a kidney function biomarker. A kidney function biomarker may be a substance or process that is indicative of the state of kidney function. A kidney function biomarker may indicate kidney impairment. A kidney function biomarker may be a molecule secreted by the kidney. A kidney function biomarker may be the result of kidney activity. A kidney function biomarker may indicate abnormal kidney function. A kidney function biomarker may be produced by a tissue or organ that is not the kidney. A kidney function biomarker may be creatinine.

### 1). Creatinine

The methods disclosed herein may detect creatinine. Creatinine may be a breakdown product of creatinine phosphate in muscle. Serum creatinine may be a byproduct of muscle metabolism. Creatinine may be produced at a constant rate in the body of a subject. Creatinine may be excreted unchanged by the kidneys. Creatinine may be removed from the blood by the kidneys. Creatinine may be removed from the body by glomerular filtration. Creatinine may be removed from the body by proximal tubular secretion. Creatinine may be filtered through the kidneys. Increased levels of creatinine in serum may indicate kidney impairment. Decreased levels of creatinine in urine may indicate kidney impairment. Detecting levels of creatinine may indicate the stage of kidney impairment. Non-linear levels of creatinine in a diabetic patient may predict kidney failure. The creatinine may be measured over time, with samples being collected at different time periods. The level of creatinine may indicate, but is not limited to abnormal kidney function, kidney impairment, kidney disease, kidney failure, kidney cancer, diabetic kidney disease, polycystic kidney disease, autosomal dominant polycystic kidney disease, kidney infection, kidney cysts, kidney stones, acute kidney injury. The level of creatinine may determine the stage of kidney disease. The level of creatinine may determine the level of kidney function. Abnormal kidney function may indicate that the subject has kidney impairment, kidney disease, kidney failure, kidney cancer, diabetic kidney disease, polycystic kidney disease, autosomal dominant polycystic kidney disease, kidney infection, kidney cysts, kidney stones, acute kidney injury. The level of creatinine may determine the effectiveness of a kidney disease treatment. The level of creatinine may indicate the success of a kidney transplant. The level of creatinine may indicate the need for a kidney transplant. Biomarkers may be used for kidney disease detection, diagnosis, prognosis, stage monitoring, indicating the treatment that may be the most effective, monitoring the effectiveness of treatment, and epidemiology.

### ii. Cancer Biomarker

The methods disclosed herein may detect cancer. The methods disclosed herein may detect a cancer antigen. The methods disclosed herein may detect a cancer biomarker. A cancer biomarker may be a substance or process that is indicative of the presence of cancer in the body. A cancer biomarker may be a molecule secreted by a tumor. A biomarker may be secreted in response to the presence of cancer in the body. Biomarkers may be used for cancer detection, diagnosis, prognosis, stage monitoring, indicating the treatment that may be the most effective, monitoring the effectiveness of treatment, and epidemiology.

A cancer biomarker may include, but is not limited to HER2, ER, PR, CA 15.3, CA 27.29, CEA, AFP, BCR-ABL, BRCA1/BRCA2, BRAF V600E, CA-125, CA19.9, KIT, PSA, S100, bladder tumor antigen, thyroglobulin, alpha-fetoprotein, leptin, prolactin, osteopontin, IGF-II, CD98, fascin, troponin I, B-type natriuretic peptide, c-myc, IL-6, fibrinogen, EGFR, gastrin, PH, G-CSF, desmin, NSE, FSH, VEGF, p21, PCNA, calcitonin, LH, somastatin, insulin, alpha-prolactin, ACTH, Bc1-2, Ki-67, p53, cathespin D, beta catenin, VWF, CD15, k-ras, caspase 3, EPN, CD10, FAS, CD30L, CD30, CGA, CRP, prothrombin, CD44, APEX, transferrin, GM-CSF, E-cadherin, IL-2, Bax, IFN-gamma, beta-2-MG, TNF-alpha, c-erbB-2, trypsin, and cyclin D. In some embodiments HER2 may be used as a cancer biomarker. In some embodiments ER may be used as a cancer biomarker.

### 1). HER2

The methods disclosed herein may detect HER2. The HER family of proteins include 4 proteins including 3 heterodimers (HER1, HER3 and HER4), a homodimer (HER2). HER2 is a member of the human epidermal growth factor receptor family and is encoded by the gene ERBB2. ERBB2 is located on the long arm of human chromosome 17 (17q12). HER2 may also be known as receptor tyrosine-protein kinase erbB-2, CD340 (cluster of differentiation 340), proto-oncogene Neu, Erbb2 (rodent), and ERBB2 (human). HER2 may be found in all breast cells. HER2 may be present in breast cells at varying levels. High levels of HER2 may indicate breast cancer. High levels of HER2 may indicate breast cancer growth. High levels of HER2 may indicate breast cancer metastasis. HER2 is an oncogene, in which amplification or over-expression may play a role in the development and progression of cancer. Pathways that HER2 may play a role in include, but are not limited to mitogen-activated protein kinase (MAPK), phosphoinositide 3-kinase (PI3K/Akt), phospholipase C (PLC), protein kinase C (PKC), signal transducer and activator of transcription (STAT).

HER2 may be a biomarker of cancer. Altered levels of HER2 may indicate a subject has cancer. HER2 may be a biomarker of breast cancer. HER2 may be a biomarker of aggressive breast cancer. HER2 may be a target for treatment of breast cancer. The methods disclosed herein may detect levels of HER2. The methods disclosed herein may indicate HER2 as a target for treatment of breast cancer.

### 2). Estrogen Receptor

The methods disclosed herein may detect an estrogen receptor. There are at least two classes of estrogen receptor (ER), ERα and ERβ. In humans, the two forms of the estrogen receptor are encoded by different genes, ESR1 and ESR2 on the sixth and fourteenth chromosome (6q25.1 and 14q23.2), respectively. ER is activated by the hormone estrogen. Once activated by estrogen, ER may translocate into the nucleus and bind to DNA to regulate the activity of different genes. Altered levels of ER may indicate there is a hormonal component to the cancer. Altered levels of ER may indicate hormonal therapy would be effective in treating cancer. Altered levels of ER may indicate endocrine therapy would be effective in treating cancer.

ER may be a biomarker of cancer. Altered levels of ER may indicate a subject has cancer. ER may be a biomarker of breast cancer. ER may indicate there is a hormonal component to the cancer. ER may be a target for the treatment of breast cancer. The methods disclosed herein may detect levels of ER. The methods disclosed herein may indicate ER as a target for treatment of breast cancer.

### iii. Metabolite

The methods disclosed herein may detect a biomarker. The biomarker may be a metabolite. Metabolites are the intermediates and products of metabolism. The metabolite may be a small molecule. Metabolites have various functions, including fuel, structure, signaling, stimulatory and inhibitory effects on enzymes, catalytic activity of their own (e.g., as a cofactor to an enzyme), defense, and interactions with other organisms (e.g., pigments, odorants, and pheromones). The metabolome forms a large network of metabolic reactions, where outputs from one enzymatic chemical reaction are inputs to other chemical reactions. Metabolites from chemical compounds, whether inherent or pharmaceutical, may be formed as part of the natural biochemical process of degrading and eliminating the compounds. The rate of degradation of a compound is an important determinant of the duration and intensity of its action. The methods disclosed herein may include determining the level of a metabolite. The methods disclosed herein may include determining the level of a kidney function metabolite. The methods disclosed herein may include determining the level of a cancer metabolite.

### 1). Cancer Metabolite

The methods disclosed herein may detect a cancer metabolite. The metabolite may be an oncometabolite. The oncometabolite may initiate or sustain tumor growth and metastasis. Metabolites associated with cancer include, but are not limited to, phosphocholine, isoleucine, threonine, glutamate, histidine, acetoactetate, glycerol, mannose, pyruvate, taurine, lactate, choline, phenylalanine, isoglutamine, tyrosine, lipids, triglycerides, TCA cycle intermediates, phosphatidylcholine, lysophosphatidylcholine, phosphocholine, glycerophosphocholine, and arachidonic acid. In some embodiments, the metabolite used herein may be listed the Human Metabolome Database (HMDB).

### g. Abnormal Kidney Function

A function of the kidneys is to filter the blood and remove excess fluid from the blood. Abnormal kidney function may result from conditions that may cause damage or disease to the kidneys. Abnormal kidney function may indicate that the subject has, without being limited to, kidney impairment, kidney disease, kidney failure, kidney cancer, diabetic kidney disease, polycystic kidney disease, autosomal dominant polycystic kidney disease, kidney infection, kidney cysts, kidney stones, acute kidney injury. Abnormal kidney function may indicate that a kidney disease treatment is not working. Abnormal kidney function may indicate that a kidney transplant is not successful. Abnormal kidney function may indicate the need for a kidney transplant.

### h. Cancer

Cancer is a group of related diseases that may include sustained proliferative signaling, evasion of growth suppressors, resistance to cell death, enablement of replicative immortality, induction of angiogenesis, and the activation of invasion and metastasis. Cancer that can be detected by the disclosed methods, includes, but is not limited to, astrocytoma, adrenocortical carcinoma, appendix cancer, basal cell carcinoma, bile duct cancer, bladder cancer, bone cancer, brain cancer, brain stem glioma, breast cancer, cervical cancer, colon cancer, colorectal cancer, cutaneous T-cell lymphoma, ductal cancer, endometrial cancer, ependymoma, Ewing sarcoma, esophageal cancer, eye cancer, gallbladder cancer, gastric cancer, gastrointestinal cancer, germ cell tumor, glioma, hepatocellular cancer, histiocytosis, Hodgkin lymphoma, hypopharyngeal cancer, intraocular melanoma, Kaposi sarcoma, kidney cancer, laryngeal cancer, leukemia, liver cancer, lung cancer, lymphoma, macroglobulinemia, melanoma, mesothelioma, mouth cancer, multiple myeloma, nasopharyngeal cancer, neuroblastoma, non-Hodgkin lymphoma, osteosarcoma, ovarian cancer, pancreatic cancer, parathyroid cancer, penile cancer, pharyngeal cancer, pituitary cancer, prostate cancer, rectal cancer, renal cell cancer, retinoblastoma, rhabdomyosarcoma, sarcoma, skin cancer, small cell lung cancer, small intestine cancer, squamous cell carcinoma, stomach cancer, T-cell lymphoma, testicular cancer, throat cancer, thymoma, thyroid cancer, trophoblastic tumor, urethral cancer, uterine cancer, uterine sarcoma, vaginal cancer, vulvar cancer and Wilms tumor. In some embodiments, the cancer is breast cancer.

### i. Breast Cancer

Breast Cancer has claimed the lives of over 40000 women in the United States alone in 2015 according to the Seer Cancer Statistics and the National institutes of Health, which is second only to lung cancer in cancer morbidity and mortality in women. The methods disclosed herein may be used to detect the level of a biomarker in a subject with breast cancer. The methods disclosed may be used for the personalized treatment of breast cancer. Breast cancer may begin in the cells of the lobules, which are the milk-producing glands, or the ducts. Breast cancer may begin in the stromal tissues, which include the fatty and fibrous connective tissues of the breast. Breast cancer may metastasize. Breast cancer may have a genetic etiology. Breast cancer may not have a genetic etiology. Breast cancer may be caused by environmental factors.

There are multiple stages of breast cancer, including stage 0, IA, IB, IIA, IIB, IIIA, IIIB, IIIC, and IV. Stage 0 indicates that the cancer has not spread outside the breast and no lymph nodes are involved. Stage IA indicates that the tumor measures up to 2 cm, the cancer has not spread outside the breast, and no lymph nodes are involved. Stage IB indicates there is no tumor in the breast, but small groups of cancer cells that are larger than 0.2 millimeter but not larger than 2 millimeters are found in the lymph nodes, or there is a tumor in the breast that is no larger than 2 centimeters, and there are small groups of cancer cells that are larger than 0.2 millimeter but not larger than 2 millimeters in the lymph nodes. Stage IIA indicates that no tumor can be found in the breast, but cancer cells are found in the axillary lymph nodes (the lymph nodes under the arm), or the tumor measures 2 centimeters or smaller and has spread to the axillary lymph nodes, or the tumor is larger than 2 but no larger than 5 centimeters and has not spread to the axillary lymph nodes. Stage IIB indicates that the tumor is larger than 2 but no larger than 5 centimeters and has spread to the axillary lymph nodes, or the tumor is larger than 5 centimeters but has not spread to the axillary lymph nodes. Stage IIIA indicates that no tumor is found in the breast, but cancer is found in axillary lymph nodes that are sticking together or to other structures, or cancer may be found in lymph nodes near the breastbone, or the cancer has spread to the axillary lymph nodes, which are sticking together or to other structures, or cancer may be found in lymph nodes near the breastbone. Stage IIIB indicates that the tumor may be any size and has spread to the chest wall and/or skin of the breast, and may have spread to axillary lymph nodes that are clumped together or sticking to other structures, or cancer may have spread to lymph nodes near the breastbone. Inflammatory breast cancer is considered at least stage IIIB. Stage IIIC indicates there may either be no sign of cancer in the breast or a tumor may be any size and may have spread to the chest wall and/or the skin of the breast, and the cancer has spread to lymph nodes either above or below the collarbone, and the cancer may have spread to axillary lymph nodes or to lymph nodes near the breastbone. Stage IV indicates the cancer has spread or metastasized to other parts of the body. Determining the stage of cancer may be important for choosing treatment options.

### 3. Methods of Detecting Abnormal Kidney Function

Provided herein are methods of detecting abnormal kidney function. The methods disclosed herein may detect one or more kidney function antigens. The methods may detect one or more kidney function biomarkers. The method may include the use of a dual nanoparticle-enhanced enzymatic immune biosensor assay. The method may include the use of a single nanoparticle-enhanced enzymatic immune biosensor assay. A first antibody may be specific to a biomarker. A first antibody and second antibody may be specific to the same biomarker. The method may then include determining the level of the product and comparing the level of product to a reference level of product, thereby detecting the level of the biomarker. The biomarker may be a kidney function biomarker. The biomarker may be a metabolite. The subject may be identified as having abnormal kidney function when the level of the product is different than the level of product from a reference level of product. The methods may include determining the subject's stage of kidney disease. The methods may further include determining the subtype of the subject's kidney disease. The methods may further comprise administering one or more pharmaceutical agents.

### a. Dual Nanoparticle-Enhanced Enzymatic Immune Biosensor Assay

The dual nanoparticle-enhanced biosensor assay relies on two populations of nanoparticles. Each population of nanoparticles is conjugated to an antibody that binds to a specific antigen of interest. The first antibody and second antibody may be the same antibody. The first antibody and second antibody may be different, but bind to the same antigen. The first antibody and second antibody may bind to the same antigen, but bind a different epitope on the antigen. The second population of nanoparticles may be bound to an enzyme (e.g., invertase). The sample may be subjected to an external magnetic field, to retain the nanoparticles in the sample. The sample may be washed while subjected to the magnetic field. The nanoparticles remain in the sample, and are bound by the conjugated antibody, to sample antigen. A substrate is added to the sample. The enzyme bound to the nanoparticle will convert the substrate to a product. Addition of a substrate (e.g., sucrose) to the reaction vessel produces a product (e.g., glucose) that is directly proportional to the sample antigen present. A schematic of the dual nanoparticle enhanced biosensor assay is shown in FIG. 1. The antigen may be a biomarker. The biomarker may be a kidney function biomarker. The biomarker may be a metabolite. The metabolite may be associated with kidney function.

### b. Single Nanoparticle-Enhanced Enzymatic Immune Biosensor Assay

The single nanoparticle-enhanced biosensor assay relies on one population of nanoparticles. The nanoparticles are conjugated to antibodies that bind to an antigen of interest. An enzyme (e.g., invertase) is conjugated to an exogenously supplied specific antigen of interest. The exogenously supplied specific antigen of interest may be a control. The quantity of the enzyme-bound control antigen may be known. A sample, which may or may not contain the antigen of interest, is contacted with a) the nanoparticle conjugated to an antibody, and b) the enzyme-bound control antigen. The enzyme-bound control antigen competes with the sample antigen for binding to the nanoparticle-conjugated antibody. The sample may be subjected to an external magnetic field, to retain the nanoparticles in the sample. The sample may be washed while subjected to the magnetic field. The nanoparticles remain in the sample, and are bound by the conjugated antibody, to sample antigen and/or control antigen. A substrate is added to the reaction vessel. The enzyme bound to the control antigen will convert the substrate to a product. Addition of a substrate (e.g., sucrose) to the reaction vessel produces a product (e.g., glucose) that is inversely proportional to the sample antigen present. This value may be calculated based on a standard curve generated with known concentrations of the control antigen. A schematic of the single nanoparticle-enhanced enzymatic immune biosensor assay is shown in FIG. 6. The antigen may be a biomarker. The biomarker may be a kidney function biomarker. The biomarker may be a metabolite. The metabolite may be associated with kidney function

### c. Antibodies

Examples of antibodies that can be used with the methods disclosed herein include, but are not limited to a polyclonal antibody, a monoclonal antibody, a human antibody, an immunoglobulin molecule, a disulfide linked Fv, a monoclonal antibody, an affinity matured, a scFv, a chimeric antibody, a single domain antibody, a CDR-grafted antibody, a diabody, a humanized antibody, a multi-specific antibody, a Fab, a dual specific antibody, a DVD, a Fab′, a bispecific antibody, a F(ab′)2, a Fv, and combinations thereof. The antibody may bind to an antigen. The antibody may bind to a kidney function antigen. The antibody may bind to a biomarker. The antibody may bind to a kidney function biomarker. The antibody may bind to a kidney function biomarker. The antibody may be an anti-creatinine antibody. The first antibody and second antibody may be the same antibody. The first antibody and second antibody may be different, but bind to the same antigen. The first antibody and second antibody may bind to the same antigen at different locations on the antigen. The first antibody and the second antibody may bind to a same epitope of the antigen. The first antibody and the second antibody may bind to a different epitope of the antigen. The first and second antibody may have specificity for different epitope(s) of the same antigen. The first antibody may be an antibody raised in an animal species different from that of the first antibody. Immobilized antibodies or fragments thereof may be incorporated into the methods. The antibodies may be immobilized onto a variety of supports, such as magnetic or chromatographic matrix particles, the surface of an assay plate (such as microtiter wells), pieces of a solid substrate material, and the like. The antibody may be conjugated to a nanoparticle. The antibody may be labeled. The antibody may be conjugated to a detectable label. In some embodiments, a first antibody may be conjugated to a first nanoparticle. A second antibody may be conjugated to a second nanoparticle. A second antibody may be conjugated to a second nanoparticle. A second antibody and an enzyme may be conjugated a second nanoparticle. Antibodies may be bound to nanoparticles by covalent binding through the reaction between the amino groups in antibody with carboxylic groups in fluorinated graphene oxide.

### d. Nanoparticles

Nanoparticles may be particles between 1 and 100 nanometers in size. Methods for generating nanoparticles include, but are not limited to gas condensation, attrition, chemical precipitation, pyrolysis and hydrothermal synthesis. In attrition, macro- or micro-scale particles may be ground in a ball mill, a planetary ball mill, or other size-reducing mechanism. The surface coating of nanoparticles may determine their properties, including stability, solubility, and targeting. The nanoparticles may have magnetic properties. The nanoparticles may be immobilized onto a variety of supports, such as magnetic or chromatographic matrix particles, the surface of an assay plate (such as microtiter wells), pieces of a solid substrate material, and the like. The nanoparticles may be one or more of Fe₃O₄ nanoparticle or a graphene oxide nanoparticle. The nanoparticles may be functionalized. The nanoparticle may be functionalized with thiolated gold. The nanoparticle may be conjugated to an antibody. The nanoparticle may be conjugated to an enzyme. Antibodies may be bound to nanoparticles by covalent binding through the reaction between the amino groups in antibody with carboxylic groups in fluorinated graphene oxide.

There may be one population of nanoparticles used in the methods disclosed herein. There may be multiple populations of nanoparticles used in the methods disclosed herein. In some embodiments there may be two different populations of nanoparticles. The first nanoparticle may be a Fe₃O₄ nanoparticle functionalized with thiolated gold. The second nanoparticle may be a graphene oxide nanoparticle conjugated to thiolated gold. The first nanoparticle may be a citric acid functionalized Fe₃O₄ aqueous colloidal magnetic nanoparticle. The second nanoparticle may be a fluorinated graphene oxide-silver nanoparticle. The nanoparticle may be conjugated to an antibody. The nanoparticle may be conjugated to multiple antibodies.

### e. Detection

The method may include determining the level of the product and comparing the level of product to a reference level of product, thereby detecting the level of the antigen. The substrate may be converted to a product. The product may be converted from a substrate. The product may be converted by an enzyme. In some embodiments, the substrate may be, but is not limited to, sucrose or acetoacetic acid. In some embodiments the enzyme may be, but is not limited to, invertase or βHB-hydrogenase. In some embodiments, the product may be, but is not limited to, glucose, βhydroxybutyrate, or a ketone. In some embodiments, the substrate may be sucrose, the enzyme may be invertase, and the product may be glucose. In some embodiments, the substrate may be acetoacetic acid the enzyme may be βHB-hydrogenase, and the product may be βhydroxybutyrate.

The level of the product from the subject's sample may be compared to a standard curve. The level of product from a subject's sample may be compared to the level of product from a normal control sample. For example a normal control sample may be a sample from a healthy individual. The level of product from a subject's sample may be compared to the level of product from an abnormal control sample. For example, an abnormal sample may be from a subject with abnormal kidney function or a subject with cancer. In some embodiments, the methods may include comparing the level of product to a reference level of product. The subject may be identified as having abnormal kidney function when the level of product is different from the reference level of product.

In some embodiments, the subject is identified as having abnormal kidney function when (a) the level of glucose in the sample is different than the reference level of glucose; (b) the level of glucose in the sample is greater than the reference level of glucose; (c) the level of βhydroxybutyrate in the sample is different than the reference level of βhydroxybutyrate; or (d) the level of creatinine in the sample is greater than the reference level of βhydroxybutyrate.

In some embodiments, the methods may further include comparing the level of at least one antigen to a reference level of the at least one antigen. The subject may be identified as having abnormal kidney function when the level of the at least one antigen is different from the reference level of the at least one antigen.

In some embodiments, the subject is identified as having abnormal kidney function when (a) the level of creatinine in the sample is different than the reference level of creatinine; (b) the level of creatinine in the sample is greater than the reference level of creatinine; (c) the level of creatinine in the sample is different than the reference level of creatinine; or (d) the level of creatinine in the sample is greater than the reference level of creatinine.

In some embodiments, the sample is a biological sample. In some embodiments, the reference level of the antigen is the level of the antigen in a control sample. In some embodiments, the reference level of the antigen is a cutoff level. In some embodiments, the cutoff value is determined by a receiver operating curve (ROC) analysis from biological samples of a patient group. In some embodiments, the cutoff level is determined by a mean plus 2 standard deviation analysis of multiple control samples. In some embodiments, the subject has abnormal kidney function. In some embodiments, the methods may further comprise administering one or more pharmaceutical agents to the subject. In some embodiments, the method may further comprise administering one or more pharmaceutical agents when abnormal kidney function is detected.

### f. Abnormal Kidney Function

A function of the kidneys is to filter the blood and remove excess fluid from the blood. Abnormal kidney function may result from conditions that may cause damage or disease to the kidneys. Abnormal kidney function may indicate that the subject has, without being limited to, kidney impairment, kidney disease, kidney failure, kidney cancer, diabetic kidney disease, polycystic kidney disease, autosomal dominant polycystic kidney disease, kidney infection, kidney cysts, kidney stones, acute kidney injury. Abnormal kidney function may indicate that a kidney disease treatment is not working. Abnormal kidney function may indicate that a kidney transplant is not successful. Abnormal kidney function may indicate the need for a kidney transplant.

### g. Kidney Function Biomarker

A biomarker detected in the methods disclosed herein may be a kidney function biomarker. A kidney function biomarker may be a substance or process that is indicative of the determining the state of kidney function. A kidney function biomarker may be a molecule secreted by the kidney. A biomarker may be secreted in response to the kidney function in the body. Biomarkers include, but are not limited to genetic, epigenetic, proteomic, glycomic, and imaging biomarkers. Biomarkers may be the target of the first and second antibodies of the methods disclosed herein. Biomarkers may be assayed from samples from a patient. Biomarkers may be assayed from body fluids. Biomarkers may be assayed from blood, serum, urine, cerebrospinal fluid, amniotic fluid, saliva, or sweat. The level of a biomarker may indicate that a subject has kidney impairment, kidney disease, kidney failure, kidney cancer, diabetic kidney disease, polycystic kidney disease, autosomal dominant polycystic kidney disease, kidney infection, kidney cysts, kidney stones, or acute kidney injury. Biomarkers may be used for kidney disease detection, diagnosis, prognosis, stage monitoring, indicating the treatment that may be the most effective, monitoring the effectiveness of treatment, and epidemiology. Antibodies to be used with the methods disclosed herein may target a kidney function biomarker.

### 4. Method of Detecting Cancer

Provided herein are methods of detecting cancer. The methods may detect one or more cancer antigens. The methods may detect one or more cancer biomarkers. The method may include the use of a dual nanoparticle-enhanced enzymatic immune biosensor assay. The method may include the use of a single nanoparticle-enhanced enzymatic immune biosensor assay. A first antibody may be specific to an antigen. A first antibody and second antibody may be specific to the same antigen. The method may then include determining the level of the product and comparing the level of product to a reference level of product, thereby detecting the level of the antigen. The antigen may be a biomarker. The biomarker may be a cancer biomarker. The biomarker may be a metabolite. The subject may be identified as having cancer when the level of the product is different than the level of product from a reference level of product. The methods may include determining the subject's stage of cancer. The methods may further include determining the subtype of the subject's cancer. The methods may further comprise administering one or more pharmaceutical agents.

### a. Dual Nanoparticle-Enhanced Immune Biosensor Assay

The dual nanoparticle-enhanced biosensor assay relies on two populations of nanoparticles. Each population of nanoparticles is conjugated to an antibody that binds to a specific antigen of interest. The first antibody and second antibody may be the same antibody. The first antibody and second antibody may be different, but bind to the same antigen. The first antibody and second antibody may bind to the same antigen, but bind a different epitope on the antigen. The second population of nanoparticles may be bound to an enzyme (e.g., invertase). The sample may be subjected to an external magnetic field, to retain the nanoparticles in the sample. The sample may be washed while subjected to the magnetic field. The nanoparticles remain in the sample, and are bound by the conjugated antibody, to sample antigen. A substrate is added to the sample. The enzyme bound to the nanoparticle will convert the substrate to a product. Addition of a substrate (e.g., sucrose) to the reaction vessel produces a product (e.g., glucose) that is directly proportional to the sample antigen present. A schematic of the dual nanoparticle enhanced biosensor assay is shown in FIG. 1. The antigen may be a biomarker. The biomarker may be a cancer biomarker. The biomarker may be a metabolite. The metabolite may be associated with cancer.

### b. Single Nanoparticle-Enhanced Immune Biosensor Assay

The single nanoparticle-enhanced biosensor assay relies on one population of nanoparticles. The nanoparticles are conjugated to antibodies that bind to an antigen of interest. An enzyme (e.g., invertase) is conjugated to an exogenously supplied specific antigen of interest. The exogenously supplied specific antigen of interest may be a control. The quantity of the enzyme-bound control antigen may be known. A sample, which may or may not contain the antigen of interest, is contacted with a) the nanoparticle conjugated to an antibody, and b) the enzyme-bound control antigen. The enzyme-bound control antigen competes with the sample antigen for binding to the nanoparticle-conjugated antibody. The sample may be subjected to an external magnetic field, to retain the nanoparticles in the sample. The sample may be washed while subjected to the magnetic field. The nanoparticles remain in the sample, and are bound by the conjugated antibody, to sample antigen and/or control antigen. A substrate is added to the reaction vessel. The enzyme bound to the control antigen will convert the substrate to a product. Addition of a substrate (e.g., sucrose) to the reaction vessel produces a product (e.g., glucose) that is inversely proportional to the sample antigen present. This value may be calculated based on a standard curve generated with known concentrations of the control antigen. A schematic of the single nanoparticle-enhanced enzymatic immune biosensor assay is shown in FIG. 6. The antigen may be a biomarker. The biomarker may be a cancer biomarker. The biomarker may be a metabolite. The metabolite may be associated with cancer.

### c. Antibodies

Examples of antibodies that can be used with the methods disclosed herein include, but are not limited to a polyclonal antibody, a monoclonal antibody, a human antibody, an immunoglobulin molecule, a disulfide linked Fv, a monoclonal antibody, an affinity matured, a scFv, a chimeric antibody, a single domain antibody, a CDR-grafted antibody, a diabody, a humanized antibody, a multi-specific antibody, a Fab, a dual specific antibody, a DVD, a Fab′, a bispecific antibody, a F(ab′)2, a Fv, and combinations thereof. The antibody may bind to an antigen. The antibody may bind to a cancer antigen. The antibody may bind to a biomarker. The antibody may bind to a cancer antigen. The antibody may bind to a cancer biomarker. The antibody may be an anti-HER2 antibody. The antibody may be an anti-ER antibody. The first antibody and second antibody may be the same antibody. The first antibody and second antibody may be different, but bind to the same antigen. The first antibody and second antibody may bind to the same antigen at different locations on the antigen. The first antibody and the second antibody may bind to a same epitope of the antigen. The first antibody and the second antibody may bind to a different epitope of the antigen. The first and second antibody may have specificity for different epitope(s) of the same antigen. The first antibody may be an antibody raised in an animal species different from that of the first antibody. Immobilized antibodies or fragments thereof may be incorporated into the methods. The antibody may be immobilized onto a variety of supports, such as magnetic or chromatographic matrix particles, the surface of an assay plate (such as microtiter wells), pieces of a solid substrate material, and the like. The antibody may be conjugated to a nanoparticle. The antibody may be labeled. The antibody may be conjugated to a detectable label. In some embodiments, a first antibody may be conjugated to a first nanoparticle. A second antibody may be conjugated to a second nanoparticle. A second antibody and an enzyme may be conjugated a second nanoparticle. Antibodies may be bound to nanoparticles by covalent binding through the reaction between the amino groups in antibody with carboxylic groups in fluorinated graphene oxide.

### d. Nanoparticles

Nanoparticles may be particles between 1 and 100 nanometers in size. Methods for generating nanoparticles include, but are not limited to gas condensation, attrition, chemical precipitation, pyrolysis and hydrothermal synthesis. In attrition, macro- or micro-scale particles may be ground in a ball mill, a planetary ball mill, or other size-reducing mechanism. The surface coating of nanoparticles may determine their properties, including stability, solubility, and targeting. The nanoparticles may have magnetic properties. The nanoparticles may be immobilized onto a variety of supports, such as magnetic or chromatographic matrix particles, the surface of an assay plate (such as microtiter wells), pieces of a solid substrate material, and the like. The nanoparticles may be one or more of Fe₃O₄ nanoparticle or a graphene oxide nanoparticle. The nanoparticles may be functionalized. The nanoparticle may be functionalized with thiolated gold. The nanoparticle may be conjugated to an antibody. The nanoparticle may be conjugated to an enzyme. Antibodies may be bound to nanoparticles by covalent binding through the reaction between the amino groups in antibody with carboxylic groups in fluorinated graphene oxide.

There may be one population of nanoparticles used in the methods disclosed herein. There may be multiple populations of nanoparticles used in the methods disclosed herein. In some embodiments there may be two different populations of nanoparticles. The first nanoparticle may be a Fe₃O₄ nanoparticle functionalized with thiolated gold. The second nanoparticle may be a graphene oxide nanoparticle conjugated to thiolated gold. The first nanoparticle may be a citric acid functionalized Fe₃O₄ aqueous colloidal magnetic nanoparticle. The second nanoparticle may be a fluorinated graphene oxide-silver nanoparticle. The nanoparticle may be conjugated to an antibody. The nanoparticle may be conjugated to multiple antibodies. Antibodies may be bound to nanoparticles by covalent binding through the reaction between the amino groups in antibody with carboxylic groups in fluorinated graphene oxide.

### e. Detection

The method may include determining the level of the product and comparing the level of product to a reference level of product, thereby detecting the level of the antigen. The substrate may be converted to a product. The product may be converted from a substrate. The product may be converted by an enzyme. In some embodiments, the substrate may be, but is not limited to, sucrose or acetoacetic acid. In some embodiments the enzyme may be, but is not limited to, invertase or βHB-hydrogenase. In some embodiments, the product may be, but is not limited to, glucose, βhydroxybutyrate, or a ketone. In some embodiments, the substrate may be sucrose, the enzyme may be invertase, and the product may be glucose. In some embodiments, the substrate may be acetoacetic acid the enzyme may be βHB-hydrogenase, and the product may be βhydroxybutyrate.

The level of the product from the subject's sample may be compared to a standard curve. The level of product from a subject's sample may be compared to the level of product from a normal control sample. For example a normal control sample may be a sample from a healthy individual. The level of product from a subject's sample may be compared to the level of product from an abnormal control sample. For example, an abnormal sample may be from a subject with abnormal kidney function or a subject with cancer. In some embodiments, the methods may include comparing the level of product to a reference level of product. The subject may be identified as having cancer when the level of product is different from the reference level of product.

In some embodiments, the subject is identified as having cancer when (a) the level of glucose in the sample is different than the reference level of glucose; (b) the level of glucose in the sample is greater than the reference level of glucose; (c) the level of βhydroxybutyrate in the sample is different than the reference level of βhydroxybutyrate; or (d) the level of HER2 in the sample is greater than the reference level of βhydroxybutyrate.

In some embodiments, the subject is identified as having cancer when (a) the level of glucose in the sample is different than the reference level of glucose; (b) the level of glucose in the sample is greater than the reference level of glucose; (c) the level of βhydroxybutyrate in the sample is different than the reference level of βhydroxybutyrate; or (d) the level of ER in the sample is greater than the reference level of βhydroxybutyrate.

In some embodiments, the methods may further include comparing the level of at least one antigen to a reference level of the at least one antigen. The subject may be identified as having cancer when the level of the at least one antigen is different from the reference level of the at least one antigen.

In some embodiments, the subject is identified as having cancer when (a) the level of HER2 in the sample is different than the reference level of HER2; (b) the level of HER2 in the sample is greater than the reference level of HER2; (c) the level of ER in the sample is different than the reference level of ER; or (d) the level of ER in the sample is greater than the reference level of ER.

In some embodiments, the sample is a biological sample. In some embodiments, the reference level of the antigen is the level of the antigen in a control sample. In some embodiments, the reference level of the antigen is a cutoff level. In some embodiments, the cutoff value is determined by a receiver operating curve (ROC) analysis from biological samples of a patient group. In some embodiments, the cutoff level is determined by a mean plus 2 standard deviation analysis of multiple control samples. In some embodiments, the subject has cancer. In some embodiments, the methods may further comprise administering one or more pharmaceutical agents to the subject. In some embodiments, the methods may further comprise administering one or more a chemotherapeutic agents to the subject. In some embodiments, the methods may further comprise administering hormone therapy to the subject. In some embodiments, the methods may further comprise administering one or more pharmaceutical agents when cancer is detected.

### f. Cancer

Cancer is a group of related diseases that may include sustained proliferative signaling, evasion of growth suppressors, resistance to cell death, enablement of replicative immortality, induction of angiogenesis, and the activation of invasion and metastasis. Cancer that can be detected by the disclosed methods, includes, but is not limited to, astrocytoma, adrenocortical carcinoma, appendix cancer, basal cell carcinoma, bile duct cancer, bladder cancer, bone cancer, brain cancer, brain stem glioma, breast cancer, cervical cancer, colon cancer, colorectal cancer, cutaneous T-cell lymphoma, ductal cancer, endometrial cancer, ependymoma, Ewing sarcoma, esophageal cancer, eye cancer, gallbladder cancer, gastric cancer, gastrointestinal cancer, germ cell tumor, glioma, hepatocellular cancer, histiocytosis, Hodgkin lymphoma, hypopharyngeal cancer, intraocular melanoma, Kaposi sarcoma, kidney cancer, laryngeal cancer, leukemia, liver cancer, lung cancer, lymphoma, macroglobulinemia, melanoma, mesothelioma, mouth cancer, multiple myeloma, nasopharyngeal cancer, neuroblastoma, non-Hodgkin lymphoma, osteosarcoma, ovarian cancer, pancreatic cancer, parathyroid cancer, penile cancer, pharyngeal cancer, pituitary cancer, prostate cancer, rectal cancer, renal cell cancer, retinoblastoma, rhabdomyosarcoma, sarcoma, skin cancer, small cell lung cancer, small intestine cancer, squamous cell carcinoma, stomach cancer, T-cell lymphoma, testicular cancer, throat cancer, thymoma, thyroid cancer, trophoblastic tumor, urethral cancer, uterine cancer, uterine sarcoma, vaginal cancer, vulvar cancer and Wilms tumor. In some embodiments, the cancer is breast cancer.

### i. Breast Cancer

Breast Cancer has claimed the lives of over 40000 women in the United States alone in 2015 according to the Seer Cancer Statistics and the National institutes of Health, which is second only to lung cancer in cancer morbidity and mortality in women. The methods disclosed herein may be used to detect breast cancer. The methods disclosed may be used for the personalized treatment of breast cancer. Breast cancer may begin in the cells of the lobules, which are the milk-producing glands, or the ducts. Breast cancer may begin in the stromal tissues, which include the fatty and fibrous connective tissues of the breast. Breast cancer may metastasize. Breast cancer may have a genetic etiology. Breast cancer may not have a genetic etiology. Breast cancer may be caused by environmental factors.

There are multiple stages of breast cancer, including stage 0, IA, IB, IIA, IIB, IIIA, IIIB, IIIC, and IV. Stage 0 indicates that the cancer has not spread outside the breast and no lymph nodes are involved. Stage IA indicates that the tumor measures up to 2 cm, the cancer has not spread outside the breast, and no lymph nodes are involved. Stage IB indicates there is no tumor in the breast, but small groups of cancer cells that are larger than 0.2 millimeter but not larger than 2 millimeters are found in the lymph nodes, or there is a tumor in the breast that is no larger than 2 centimeters, and there are small groups of cancer cells that are larger than 0.2 millimeter but not larger than 2 millimeters in the lymph nodes. Stage IIA indicates that no tumor can be found in the breast, but cancer cells are found in the axillary lymph nodes (the lymph nodes under the arm), or the tumor measures 2 centimeters or smaller and has spread to the axillary lymph nodes, or the tumor is larger than 2 but no larger than 5 centimeters and has not spread to the axillary lymph nodes. Stage IIB indicates that the tumor is larger than 2 but no larger than 5 centimeters and has spread to the axillary lymph nodes, or the tumor is larger than 5 centimeters but has not spread to the axillary lymph nodes. Stage IIIA indicates that no tumor is found in the breast, but cancer is found in axillary lymph nodes that are sticking together or to other structures, or cancer may be found in lymph nodes near the breastbone, or the cancer has spread to the axillary lymph nodes, which are sticking together or to other structures, or cancer may be found in lymph nodes near the breastbone. Stage IIIB indicates that the tumor may be any size and has spread to the chest wall and/or skin of the breast, and may have spread to axillary lymph nodes that are clumped together or sticking to other structures, or cancer may have spread to lymph nodes near the breastbone. Inflammatory breast cancer is considered at least stage IIIB. Stage IIIC indicates there may either be no sign of cancer in the breast or a tumor may be any size and may have spread to the chest wall and/or the skin of the breast, and the cancer has spread to lymph nodes either above or below the collarbone, and the cancer may have spread to axillary lymph nodes or to lymph nodes near the breastbone. Stage IV indicates the cancer has spread or metastasized to other parts of the body. Determining the stage of cancer may be important for choosing treatment options.

### g. Cancer Biomarker

A biomarker detected in the methods disclosed herein may be a cancer biomarker. A cancer biomarker may be a substance or process that is indicative of the presence of cancer in the body. A cancer biomarker may be a molecule secreted by a tumor. A biomarker may be secreted in response to the presence of cancer in the body. Biomarkers include, but are not limited to genetic, epigenetic, proteomic, glycomic, and imaging biomarkers. Biomarkers may be used for cancer detection, diagnosis, prognosis, stage monitoring, indicating the treatment that may be the most effective, monitoring the effectiveness of treatment, and epidemiology. Biomarkers may be the target of the first and second antibodies of the methods disclosed herein. Biomarkers may be assayed from samples from a patient. Biomarkers may be assayed from body fluids. Biomarkers may be assayed from a tumor cell lysate, a pre-surgical biopsy, a biopsy obtained during surgery, and/or a post-surgical biopsy. The level of a biomarker may determine the stage of breast cancer in a subject.

Cancer biomarkers include, but are not limited to HER2, ER, PR, CA 15.3, CA 27.29, CEA, AFP, BCR-ABL, BRCA1/BRCA2, BRAF V600E, CA-125, CA19.9, KIT, PSA, S100, bladder tumor antigen, thyroglobulin, alpha-fetoprotein, leptin, prolactin, osteopontin, IGF-II, CD98, fascin, troponin I, B-type natriuretic peptide, c-myc, IL-6, fibrinogen, EGFR, gastrin, PH, G-CSF, desmin, NSE, FSH, VEGF, p21, PCNA, calcitonin, LH, somastatin, insulin, alpha-prolactin, ACTH, Bc1-2, Ki-67, p53, cathespin D, beta catenin, VWF, CD15, k-ras, caspase 3, EPN, CD10, FAS, CD30L, CD30, CGA, CRP, prothrombin, CD44, APEX, transferrin, GM-CSF, E-cadherin, IL-2, Bax, IFN-gamma, beta-2-MG, TNF-alpha, c-erbB-2, trypsin, and cyclin D. In some embodiments HER2 may be used as a cancer biomarker. In some embodiments ER may be used as a cancer biomarker.

### i. HER2

The HER family of proteins include 4 proteins including 3 heterodimers (HER1, HER3 and HER4), a homodimer (HER2). HER2 is a member of the human epidermal growth factor receptor family and is encoded by the gene ERBB2. ERBB2 is located on the long arm of human chromosome 17 (17q12). HER2 may also be known as receptor tyrosine-protein kinase erbB-2, CD340 (cluster of differentiation 340), proto-oncogene Neu, Erbb2 (rodent), and ERBB2 (human). HER2 may be found in all breast cells. HER2 may be present in breast cells at varying levels. High levels of HER2 may indicate breast cancer. High levels of HER2 may indicate breast cancer growth. High levels of HER2 may indicate breast cancer metastasis. HER2 is an oncogene, in which amplification or over-expression may play a role in the development and progression of cancer. Pathways that HER2 may play a role in include, but are not limited to mitogen-activated protein kinase (MAPK), phosphoinositide 3-kinase (PI3K/Akt), phospholipase C (PLC), protein kinase C (PKC), signal transducer and activator of transcription (STAT).

HER2 may be a biomarker of cancer. Altered levels of HER2 may indicate a subject has cancer. HER2 may be a biomarker of breast cancer. HER2 may be a biomarker of aggressive breast cancer. HER2 may be a target for treatment of breast cancer. The methods disclosed herein may detect levels of HER2. The methods disclosed herein may indicate HER2 as a target for treatment of breast cancer.

### ii. Estrogen Receptor

There are at least two classes of estrogen receptor (ER), ERα and ERβ. In humans, the two forms of the estrogen receptor are encoded by different genes, ESR1 and ESR2 on the sixth and fourteenth chromosome (6q25.1 and 14q23.2), respectively. ER is activated by the hormone estrogen. Once activated by estrogen, ER may translocate into the nucleus and bind to DNA to regulate the activity of different genes. Altered levels of ER may indicate there is a hormonal component to the cancer. Altered levels of ER may indicate hormonal therapy would be effective in treating cancer. Altered levels of ER may indicate endocrine therapy would be effective in treating cancer.

ER may be a biomarker of cancer. Altered levels of ER may indicate a subject has cancer. ER may be a biomarker of breast cancer. ER may indicate there is a hormonal component to the cancer. ER may be a target for the treatment of breast cancer. The methods disclosed herein may detect levels of ER. The methods disclosed herein may indicate ER as a target for treatment of breast cancer.

### 4. Kits

Also provided herein is a kit for use in performing the methods described herein. In some embodiments, the kit may be for detecting an antigen in a sample. In some embodiments, the kit may be for detecting a biomarker in a sample. In some embodiments, the kit may be for detecting abnormal kidney function in a subject. In some embodiments, the kit may be for determining the stage of kidney disease. In some embodiments, the kit may be for detecting cancer in a subject. The kit may be for detecting breast cancer. In some embodiments, the kit may be for determining the stage of cancer. The kit may include instructions for detecting at least one antigen. The kit may include instructions for detecting at least one biomarker. Instructions included in the kit may be affixed to packaging material or may be included as a package insert. The instructions may be written or printed materials, but are not limited to such. Any medium capable of storing such instructions and communicating them to an end user is contemplated by this disclosure. Such media include, but are not limited to, smart phones, electronic storage media (e.g., magnetic discs, tapes, cartridges, chips), optical media (e.g., CD ROM), and the like. As used herein, the term “instructions” may include the address of an internet site that provides the instructions.

The kit may include one or more reagents for detecting an antigen. The kit may include one or more reagents for detecting a biomarker. In some embodiments, the kit includes one or more reagents for detecting a biomarker selected from creatinine, HER2, and ER. The kit may include reagents capable of specifically binding to each biomarker and/or to quantify the levels of the biomarker in a sample.

The kit may include nanoparticles as described herein. The kit may include antibodies as described herein. The kit may include enzymes as described herein. The kit may include substrates capable of being converted to a product by an enzyme as described herein. The kit may include a glucose meter (glucometer).

The kit may include a reference standard indicating reference levels of each of the antigens. The kit may include instructions for generating a standard curve or a reference standard for purposes of quantifying an antigen.

Optionally, the kit includes quality control components (for example, sensitivity panels, calibrators, and positive controls). Preparation of quality control reagents is well-known in the art and is described on insert sheets for a variety of diagnostic products. Sensitivity panel members optionally are used to establish assay performance characteristics, and further optionally are useful indicators of the integrity of the kit reagents, and the standardization of assays.

The kit may also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also may be included in the kit. The kit may additionally include one or more other controls. One or more of the components of the kit may be lyophilized, in which case the kit may further comprise reagents suitable for the reconstitution of the lyophilized components.

The kit may also comprise one or more containers, such as vials or bottles, with each container containing a separate reagent. The various components of the kit optionally are provided in suitable containers as necessary, e.g., a microtiter plate. The kit may further include containers for holding or storing a sample (e.g., a container or cartridge for a blood sample). Where appropriate, the kit optionally also may contain reaction vessels, mixing vessels, and other components that facilitate the preparation of reagents or the test sample. The kit may also include one or more instrument for assisting with obtaining a test sample, such as a syringe, pipette, forceps, measured spoon, or the like.

In some embodiments, the kit comprises a first antigen specific antibody conjugated to a first nanoparticle, a second antigen specific antibody conjugated to a second nanoparticle enzyme, a substrate capable of being converted to a product by the enzyme. In some embodiments, the kit comprises an antigen specific antibody conjugated to a nanoparticle, an antigen conjugated to an enzyme, and a substrate capable of being converted to a product by the enzyme.

### 6. EXAMPLES

### Example 1

### Designing a Dual Nanoparticle Hardware System

Using two different sets of nanoparticles: (i) magnetic (Fe₃O₄) nanoparticles functionalized with thiolated gold (Au), and (ii) graphene oxide nanoparticles conjugated to thiolated Au. Studies with HER2 and ER can be conducted with these nanoparticles. The dual nanoparticle system comprised of citric acid functionalized magnetic nanoparticles and fluorinated graphene-silver oxide NPs showed the lowest detection limit for the glucometer, which coincided with 22 ng/ml of HER2 indicating a 10-fold higher sensitivity of HER2 detection than the commercial sandwich ELISA for HER2. The detection of HER2 has been subject of intense investigation due to a number of issues relating to its detection. In an effort to keep the nanoparticle system constant for both HER2 and ER antigens and increase the robustness of the system for detection of both of these antigens, additional nanoparticles may be engineered.

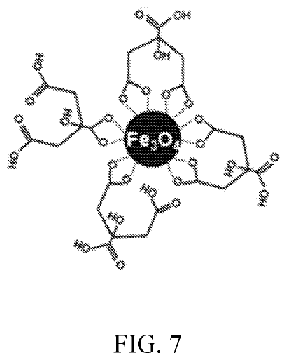

Citric acid functionalized (citrate-stabilized) Fe₃O₄ aqueous colloidal magnetic nanoparticles, which may be synthesized FIG. 7. The major advantages of these nanoparticles are that (i) they can be prepared using a facile single-step process, (ii) they are highly biocompatible and the process is reproducible, and (iii) lastly by eliminating gold conjugation will decrease cost of cancer detection assay. It is also expected that the citric acid-functionalized magnetic nanoparticles will bind to higher amounts of antibodies (Ab1) thus increasing surface area of reaction platform and would contribute to increased sensitivity of the cancer detection assay.

Fluorinated graphene oxide-silver nanoparticles (FGO-Ag NPs) may be prepared. These NPs are expected to develop the recognition layer with unique properties of FGO—carboxyl, epoxy, and hydroxyl functional groups on its basal planes and edges.

The presence of FGO would impede biofouling due to the low interfacial energy between a surface and water. The fluorine atoms modify the electronic properties of graphene by reducing the charge in the conducting π orbitals. Hence, low level inhomogeneous doping of highly electronegative fluorine can induce partial positive surfaces on graphene. The hydrophobic centers in FGO (or fluorinated graphene in the case of reduced FGO) may also act as energetic barriers between interfaces, such as proteins. Thus, FGO with its enhanced anti-biofouling ability, resulting in a more specified recognition interface, will increase the sensitivity of the developed immunosensor by decreasing the background signal. It is expected that these nanoparticles may increase sensitivity and specificity of breast cancer detection assays.

### Example 2

### Single-Step Preparation of PDDA-AuNPs

Iron oxide magnetic nanocomposites (Fe₃O₄), containing Poly(diallyl dimethylammonium) chloride (PDDA), gold nanoparticles and creatinine antibody were prepared. The first step of the nanocomposite was the preparation of PDDA-AuNPs, which were prepared by a simple, one-step approach. PDDA was purchased from Aldrich and HAuC14 and NaOH from. 250 μL PDDA (4 wt % in water), 40 mL water, 200 mL 0.5 M NaOH and 100 mL HAuCl4 (10 mg/mL) were added into a beaker. After thoroughly mixing for 2 min, the mixed solution maintained at 100 C for several minutes until the color of the solution changed to red and no further color change occurred.

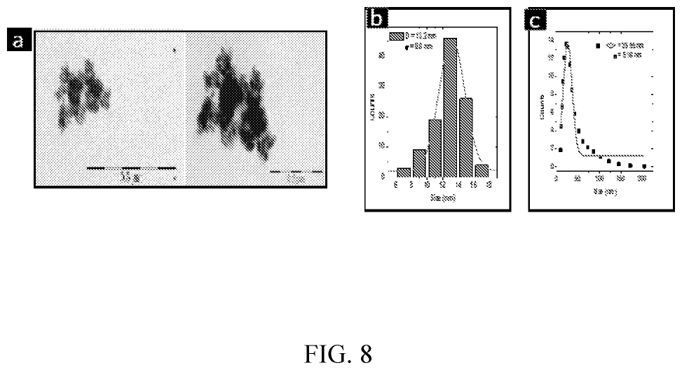

The preparation of gold-polyelectrolyte nanocomposites was a simple, single step approach, where PDDA acts as a reducing and stabilizing agent. The wine-red colloidal suspension of AuNPs was evident within two minutes of the start of the experiment. FIG. 8(a) shows a Transmission Electron Microscopy (TEM) image of the product. The images show that the AuNPs are abundantly attached to the surface of the polymer. To confirm and characterize the size of AuNPs, TEM was used to analyze the AuNPs synthesized. FIG. 8(b) shows the size distribution histograms of the AuNPs. It reveals that the average diameter of AuNPs is to be around 13 nm with a narrow size distribution. FIG. 8(c) shows the size distribution of the composite measured by the Zetatrac. It shows that the PDDA-AuNPs composite has an average size of around 26 nm.

### Example 3

### Iron Oxide Magnetic Nanoparticles with PDDA-AuNPs

Preparation of Fe₃O₄—Au-Ab bioconjugate Fe₃O₄ and anti-creatinine antibody were purchased from, and, respectively. The previously prepared PDDA-AuNPs were added to the Fe₃O₄ nanoparticles with stirring. The final Fe₃O₄ nanocomposites were washed, then separated using an external magnetic field. 1 mL of 100 μg/mL primary anti-creatinine antibody (Ab) was added to 1 ml of the Fe3O4—Au dispersion and the mixture was shaken overnight at 4° C. The reaction mixture was magnetically separated and washed with phosphate-buffered saline. The final product Fe₃O₄—Au bioconjugate was suspended in 1 mL of PBS containing 1% BSA.

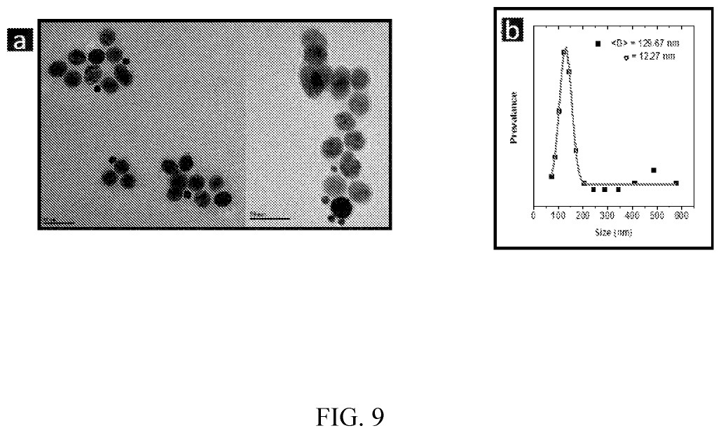

The morphology of fabricated Fe₃O₄—Au nanocomposite was studied by transmission electron microscopy (TEM). The images FIG. 9 (a) show that Fe₃O₄ was successfully adhered on the surface of the polymer. It is evident that polymer has two different particles of different sizes adhered to its surface. The graph shows the size distribution of the Fe₃O₄—Au nanocomposite, which has an average size of around 130 nm and a narrow size distribution. The results are shown in FIG. 9.

### Example 4

### Characterization of Nanoparticles

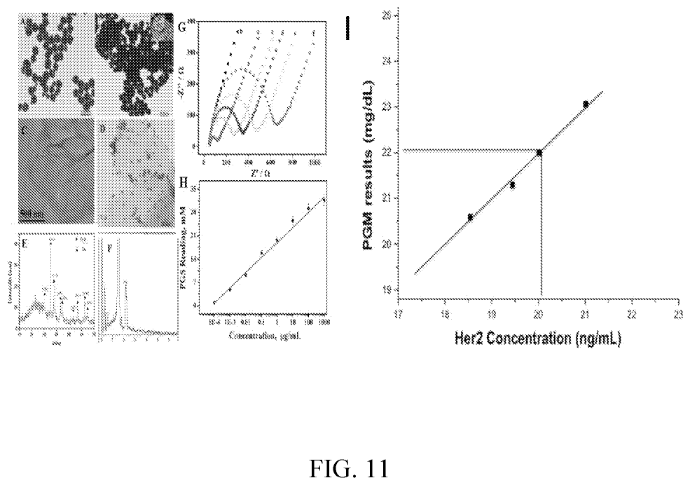

The characterization of the nanoparticles and their physico-chemical properties are shown in FIG. 11. Prior to testing for antigen detection both magnetic-gold and graphene-gold nanoparticles were characterized by transmission electron microscopy and then by scanning electron microscope. The particles were characterized by XRD and ERD to ascertain their properties. In addition, the electrochemical impedance spectra were recorded (FIG. 11G). The results showed a gradual change in the impedance. A calibration curve of the immunosensor using different concentrations of albumin was produced. The results showed that albumin is detectable using the DNEI strategy in a linear range and in a very sensitive manner. Taken together these results demonstrate the potential of the DNEI assay for detection of breast cancer antigens such as HER2 and ER. Thus, biopsy or surgical tumor tissue lysates can be analyzed using this assay, and the results can provide a more accurate and quantitative cancer detection assay of ER and HER2. These results can also assist the design of a BdT assay with appropriate drugs to predict the efficacy of those drugs in an individual patient.

### Example 5

### Developing an Antibody-Enzyme Detection Software System

The magnetic NPs are conjugated with antibodies (Ab1) with specificity to certain epitope(s) of the desired antigen and the detecting nanoparticles are conjugated with a second antibody (Ab2), which has specificity for different epitope(s) of the same antigen and is an antibody raised in an animal species different from that of the Ab1. Also, Ab2 is conjugated with an enzyme of interest, such as invertase that converts sucrose to glucose or βHB-dehydrogenase that converts acetoacetic acid to βHydroxy butyrate (Ketone). Both glucose and ketones are detectable by glucose meters. Some of the best characterized antibodies for HER2 and ER known specifically for IHC may be used.

The experiments may be conducted to optimize the amount of Ab1, the amount of ER or HER2 antigen and the amount of Ab2, the amount of substrate and the amount of enzyme needed, a dose response study may be conducted to generate a calibration curve and sensitivity and specificity test. Once the calibration curve is established, the levels of ER or HER2 may be evaluated for sensitivity in DNEI assay using lysates of MCF7 and BT494 cell lysates that express respectively, ER and HER2. BT474 cells that express high levels of HER2 with MCF7 cells that express very little or no HER2. The results may be compared for sensitivity and specificity with a classical sandwich ELISA assay.

Adaptations may be made that will make it possible to easily read and transmit HER2 and ER expression info from immunosensing to quantifiable platform. A smartphone-coupled to a glucose sensor will measure the HER2/ER expression with digital output and integrated by a smartphone application (app). It is expected that the DNEI assay will exhibit significant increase in sensitivity and specificity with a fewer microliters of samples needed for the assay. It is expected that the results of these studies will lead to the development of a cancer detection assay that will indicate the level of expression of ER or HER2 in tumor cell lysates of biopsies and tissue obtained from surgery.

### Example 6

### Iron Oxide Magnetic Nanocomposite With Creatinine Antibody

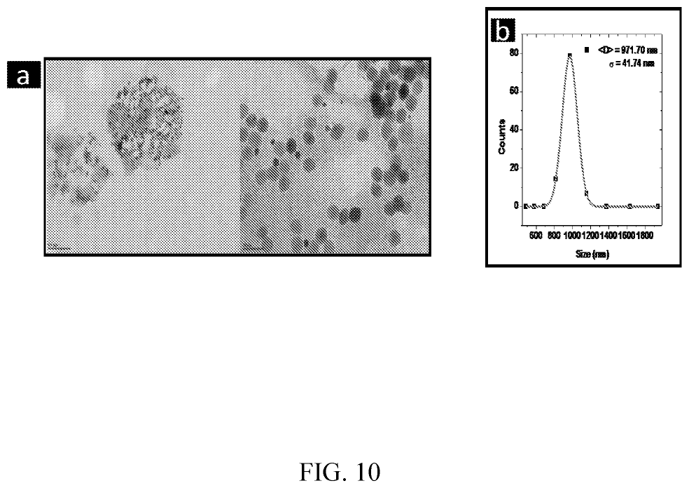

The morphology of fabricated Fe₃O₄—Au-Ab bioconjugate was studied by transmission electron microscopy (TEM). The images show a change in the morphology of the nanocomposites upon the conjugation of the antibody. The graph shows the size distribution of the Fe₃O₄—Au-Ab Bioconjugate with an average size of around 970 nm and a narrow size distribution. The increase of size further confirms that the antibody was successfully conjugated. The results are shown in FIG. 10.

### Example 7

### Measurement of IGF-1 in Fluid

The protocol for the biosensing process may be defined by three components. First, iron oxide/FGO core/shell nanoparticles are prepared by a facile and green approach for encapsulated iron oxide nanoparticles with positive charge by FGO with negative charge through electrostatic self-assembly in water solution. The resulted nanoparticles may be thoroughly characterized, such as SEM, TEM, HRTEM, DLS, etc. Then the obtained nanoparticles may be used for covalent binding primary antibody through the reaction between the amino groups in antibody with carboxylic groups in FGO. Thereafter, IGF-1 enrichment from small amount of biofluids can be achieved by incubating antibody loaded core/shell nanoparticles for certain time following effective magnetic separation. Secondly, GO-Au nanocomposites are prepared by a simple directed mixing method through electrostatic interaction. The loaded Au nanoparticles supply good biocompatible surface to cargo labeled antibody acting as trace tag. Finally, iron oxide/FGO nanoparticles enriching IGF-1 and the trace tag will complete the recognition process based on the specified interaction between antibody and antigen on the working electrode. Following silver enhancing process, the amplified electrochemical signal can be read to reveal the precise IGF-1 concentration.

These results demonstrate the potential of the DNEI assay for detection of breast cancer antigens such as HER2 and ER. Thus, biopsy or surgical tumor tissue lysates can be analyzed using this assay and the results can provide a more accurate and quantitative cancer detection assay of ER and HER2. These results can also assist the design of a BdT assay with appropriate drugs to predict the efficacy of those drugs in an individual patient.

### Example 8

### Control of Detecting Sensitivity

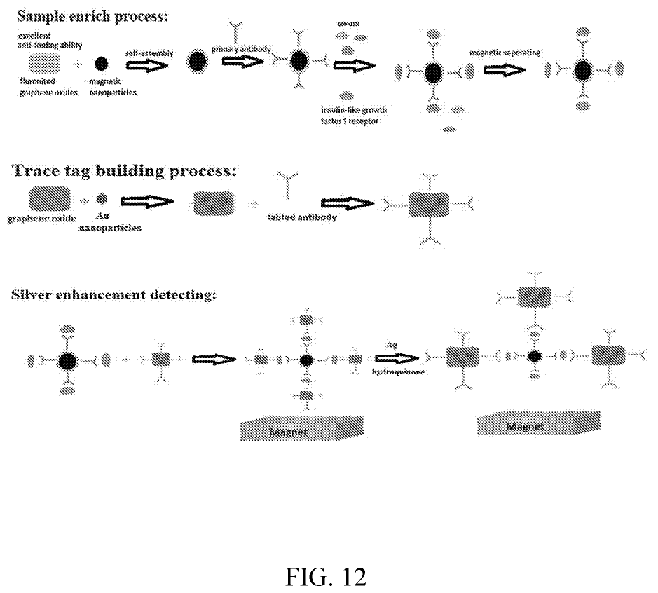

The detecting sensitivity may be controlled in the following three ways: 1) build a high anti-fouling bio-interface for protein-protein reorganization based on FGO due to the rich amount of existing C—F bonds, which can significantly reduce the detecting background, thus increase the sensitivity; 2) employ a novel iron oxide/FGO core/shell nanoparticle can capture a large amount of primary antibodies (Ab1) and thus amplify the detection response. Besides, this kind of nanoparticle can be effectively used to enrich IGF-1 in biofluids for signal enhancement; 3) adopt graphene oxide-Au hybrid nanoparticles as trace tag for signal amplification based on silver enhancement strategy by depositing silver on the gold surface (FIG. 12). The sensitivity of the biosensor can be remarkably increased by this strategy. This approach not only increases the sensitivity but also avoids the usage of enzyme, which will retain more robust detecting way.

### Example 9

### Electrochemical Sensor Approach

The overall plan involves developing highly reproducible method enabling reliable, detailed dynamical concentration in ISF or other biofluids for IGF-1 to be generated. The biosensing process can be illustrated as the following scheme (FIG. 12). The detecting sensitivity may be increased in three ways: 1) build a high anti-fouling bio-interface for protein-protein reorganization based on fluorinated graphene oxide due to the rich amount C—F bonds existing, which can significantly reduce the detecting background, thus increase the sensitivity; 2) employ a novel iron oxide/FGO core/shell nanoparticle can capture a large amount of primary antibodies (Ab1) and thus amplify the detection response. 3) Employ a novel magneto-immune sensor (MIS) approach to detect IGF-1. Besides, this kind of nanoparticle can be effectively used to enrich IGF-1 in biofluids for signal enhancement; 3) adopt graphene oxide-Au hybrid nanoparticles as trace tag for signal amplification based on silver enhancement strategy by depositing silver on the gold surface. The sensitivity of the biosensor can be remarkably increased by this strategy. This approach not only increases the sensitivity but also avoids the usage of enzyme, which will retain capacity for more robust detection.

### Example 10

### Nanoparticle-Based Sandwich Assay

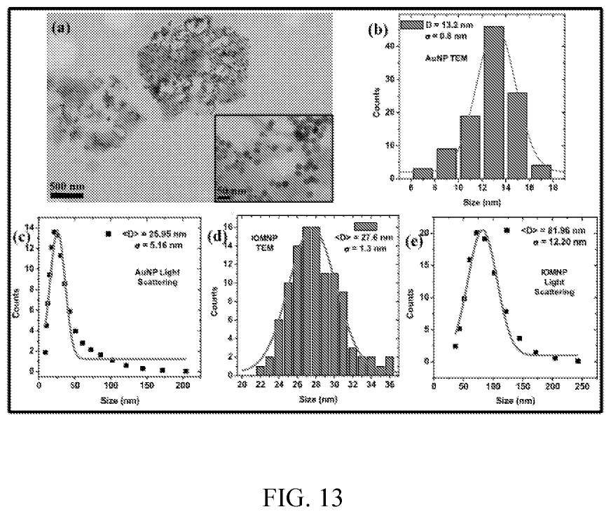

Sandwich Immunoassay. FIG. 13 describes the immunosensor's first step which is composed of iron oxide magnetic nanoparticles (IOMNPs) for magnetic separation, PDDA functionalization, and gold nanoparticles (AuNPs) for conjugation of primary antibodies (Ab-1 Construction begins with the mixing of chloroauric acid and PDDA while heating until solution color change.

The results are shown in FIG. 13. AuNPs are approximately 13 nm as measured by (a) TEM and 26 nm measured by (b) light scattering. The difference is attributed to the surrounding PDDA layer and hydration shell. After centrifugation the product is functionalized to IOMNPs with mixing. Purchased IOMNPs are approximately 28 nm in diameter as measured by (c) TEM and 82 nm measured by (d) light scattering. The difference is attributed to in situ agglomeration and hydration shells. Finally, Ab-1 is attached to the AuNPs with mixing.

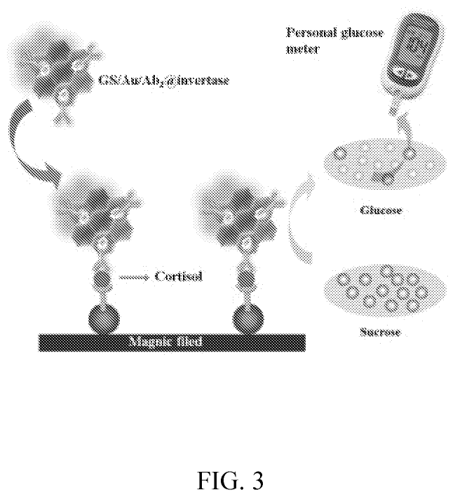

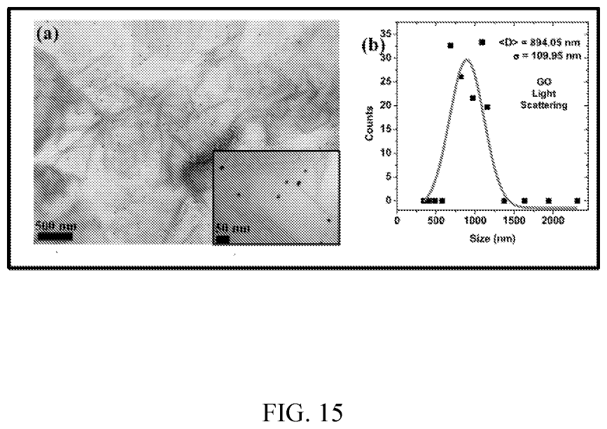

The second step of sandwich assay which is composed of graphene oxide (GO) for scaffold, PDDA for functionalization, AuNPs for conjugation of secondary antibody (Ab-2), and invertase. FIG. 3 shows invertase and cortisol binding to a nanoparticle. The GO, with 894 nm diameter measured by (i) light scattering, was mixed with PDDA-AuNP. Then invertase and Ab-2 were conjugated to AuNPs with mixing. The results are shown in FIG. 15.

This sandwich immunosensor functions through creatinine binding to Ab-1 on the IOMNP composite as well as Ab-2 on the GO composite. Magnetic field retains the ‘sandwich’ upon washing thus removing unbound invertase. Addition of sucrose to retained invertase produces glucose in proportion to creatinine.

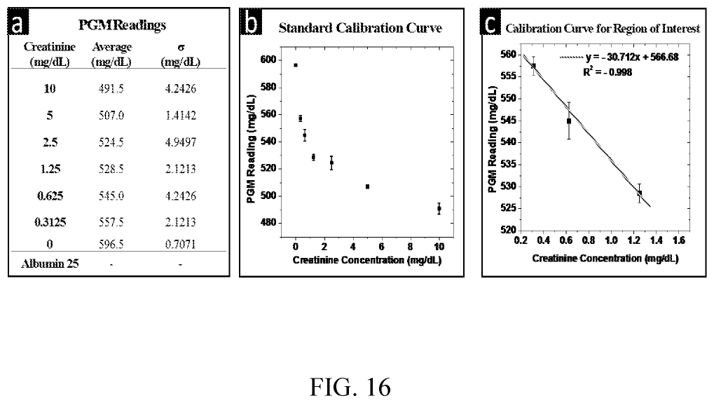

To test the performance of the creatinine system, the absence of specific reagents was tested to confirm their functionality. The absence of the creatinine-invertase conjugate gave no reading using the PGM, since sucrose was not converted to glucose in this sample. The absence of creatinine gave a high reading of almost 500 mg/dL. The lower the concentration of creatinine the higher the reading on the PGM. Each concentration was sampled twice and the PGM readings were tabulated and the average reading for each concentration was plotted to formed a standard curve shown in FIG. 16.

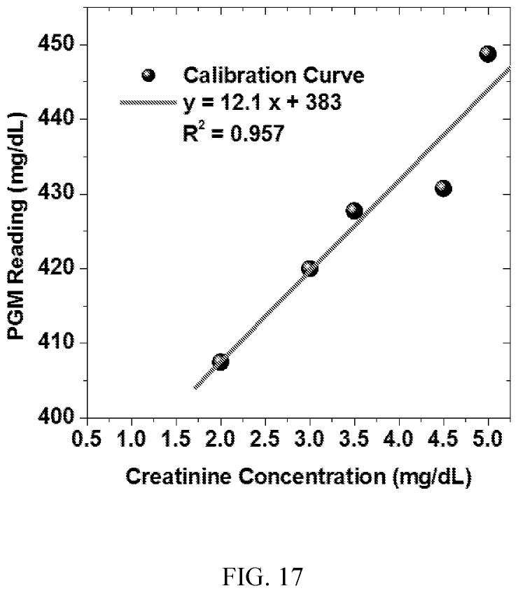

Relatively high glucose level of the positive control, no IOMNP composite and no washing performed, reflects sucrose is converted to glucose upon reaction with invertase. A relatively low glucose level of the negative control, no creatinine and washing performed, is consistent with no glucose generated. Samples containing higher concentrations of creatinine exhibit correspondingly high concentrations of glucose. For given samples, the glucose measurement is the average of at least three readings and the error determined from their standard deviation. FIG. 17 shows a Calibration of PGM with standardized glucose and creatinine samples was conducted in the range of physiologic levels of creatinine seen in serum, which was correlated (R2=0.957) with glucose detection by glucometer. Table 1 summarizes the data with different concentrations of creatinine measured between 2 to 5 mgs per deciliter.

The PGM reading increased with the decrease of creatinine concentration, since the concentration of glucose is inversely proportional to the concentration of creatinine in the sample, and directly proportional to the concentration of the creatinine-invertase conjugate. The results are shown in FIG. 16.

### Example 11

### Competitive Immunoassay

Conjugation of Creatinine-Invertase. Creatinine and invertase were purchased. To 20 mg/ml invertase in PBS, 5 mg creatinine was added and the mixture was mixed at room temperature for 4 hours.

Immunoassay. A competitive immunoassay is validated by adding the samples containing creatinine and creatinine-invertase conjugates to wells containing 50 μL IOMNPs with the creatinine antibody, which were used as the sensor for each test. When performing each test, 50 μL of creatinine (of known concentrations) and 50 μL of the prepared creatinine-invertase conjugate were mixed and added to the wells. During an incubation of 30 minutes at room temperature, the antibody competitively binds to creatinine in the sample or conjugate. The plate is washed, while applying an external magnetic field, leaving only bound creatinine and bound creatinine-invertase conjugates. To each well, 50 μL of 0.5 M sucrose solution was added. The plate was left on the roller for 2 hours before quantifying the glucose concentration. 5 μL of each solution was tested by a PGM. To determine a standard curve, creatinine was serially diluted in assay buffer and run in the assay.

A key to translate the binding event between the target (creatinine) and its antibody into a PGM-detectable signal may be by the use of a creatinine-invertase conjugate. The concentration of creatinine-invertase conjugate bound to the magnetic nanoparticles is dependent and inversely proportional to the concentration of the target in the sample, thus the concertation of glucose from the breakdown of sucrose by invertase can be used to quantitatively measure glucose and indirectly quantify creatinine concertation. To be able to detect abnormal creatinine levels at an early stage, allows for an early diagnosis of kidney malfunction. To be able to easily and non-invasively detect the levels of creatinine in urine is diagnostically favorable. PGM are sold for reasonable prices, compact and portable, and with the advent of cell-phone-based personal glucose meters, patients can electronically save their test results and even wirelessly transmit them to their doctor. This experiment can be expanded to measure other non-glucose targets.

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense. It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the present disclosure are set out in the following numbered clauses:

Clause 1. A method of detecting an antigen in a subject, the method comprising:
a) contacting a sample from the subject with:
   i) a first antibody conjugated to a first nanoparticle;
   ii) a second antibody conjugated to a second nanoparticle, wherein the nanoparticle is conjugated to an enzyme, wherein the first and second antibodies bind an antigen; and
   iii) a substrate capable of being converted to a product by the enzyme;
b) determining the level of the product;
(c) comparing the level of product to a reference level of product; thereby detecting the level of antigen.

Clause 2. The method of clause 1, wherein the first antibody and the second antibody bind to a same epitope of the antigen.

Clause 3. The method of clause 1, wherein the first antibody and the second antibody bind to a different epitope of the antigen.

Clause 4. The method of clause 1, wherein the sample is selected from blood, serum, urine, cerebrospinal fluid, amniotic fluid, saliva, sweat, a tumor cell lysate, a pre-surgical biopsy, a biopsy obtained during surgery, and a post-surgical biopsy.

Clause 5. The method of clause 1, wherein the antigen is a biomarker.

Clause 6. The method of clause 5, wherein the biomarker is a kidney function biomarker.

Clause 7. The method of clause 6, wherein the kidney function biomarker is creatinine.

Clause 8. The method of clause 1, wherein the substrate is sucrose, the enzyme is invertase, and the product is glucose.

Clause 9. The method of clause 1, wherein the substrate is acetoacetic acid, the enzyme is βHB-hydrogenase, and the product is βhydroxybutyrate.

Clause 10. The method of clause 1, wherein the level of product is detected by a glucose meter.

Clause 11. The method of clause 1, wherein the glucose meter is integrated with a smart phone application.

Clause 12. The method of clause 1, wherein the first nanoparticle is a Fe₃O₄ nanoparticle functionalized with thiolated gold.

Clause 13. The method of clause 1, wherein the first nanoparticle is a Fe₃O₄ nanoparticle functionalized with thiolated gold.

Clause 14. The method of clause 1, wherein the second nanoparticle is a graphene oxide nanoparticle conjugated to thiolated gold.

Clause 15. The method of clause 1, wherein the first nanoparticle is a citric acid functionalized Fe₃O₄ aqueous colloidal magnetic nanoparticle.

Clause 16. The method of clause 1, wherein the second nanoparticle is a fluorinated graphene oxide-silver nanoparticle.

Clause 17. A kit for detecting an antigen in a subject, the kit comprising:
a) a first antibody conjugated to a first nanoparticle;
b) a second antibody conjugated to a second nanoparticle, wherein the nanoparticle is conjugated to an enzyme, wherein the first and second antibodies bind an antigen; and
c) a substrate capable of being converted to a product by the enzyme.

Clause 18. The kit of clause 17, further comprising a glucose meter.

Clause 19. The kit of clause 17, wherein the antigen is a biomarker.

Clause 20. The kit of clause 19, wherein the biomarker is a kidney function biomarker.

Clause 21. The kit of clause 20, wherein the kidney function biomarker is creatinine.

Clause 22. The kit of clause 17, wherein the substrate is sucrose, the enzyme is invertase, and the product is glucose.

Clause 23. The kit of clause 17, wherein the wherein the substrate is acetoacetic acid, the enzyme is βHB-hydrogenase, and the product is βhydroxybutyrate.

Clause 24. The kit of clause 18, wherein the glucose meter is integrated with a smart phone application.

Clause 25. The kit of clause 17, wherein the first nanoparticle is a Fe₃O₄ nanoparticle functionalized with thiolated gold.

Clause 26. The kit of clause 17, wherein the second nanoparticle is a graphene oxide nanoparticle conjugated to thiolated gold.

Clause 27. The kit of clause 17, wherein the first nanoparticle is a citric acid functionalized Fe₃O₄ aqueous colloidal magnetic nanoparticle.

Clause 28. The kit of clause 17, wherein the second nanoparticle is a fluorinated graphene oxide-silver nanoparticle.

Clause 29. A method of detecting abnormal kidney function in a subject, the method comprising:
a) obtaining a sample from the subject;
b) contacting the sample with:
   i) a first antibody conjugated to a first nanoparticle;
   ii) a second antibody conjugated to a second nanoparticle, wherein the nanoparticle is conjugated to an enzyme, wherein the first and second antibodies bind an antigen; and
   iii) a substrate capable of being converted to a product by the enzyme
c) determining the level of the product;
d) comparing the level of product to a reference level of product; and
e) identifying the subject as having abnormal kidney function when the level of the product is different than the level of product from a reference level of product.

Clause 30. The method of clause 29, wherein the sample is selected from blood, serum, urine, sweat, and saliva.

Clause 31. The method of clause 29, wherein the abnormal kidney function indicates the subject has kidney impairment, kidney disease, kidney failure, kidney cancer, diabetic kidney disease, polycystic kidney disease, autosomal dominant polycystic kidney disease, kidney infection, kidney cysts, kidney stones, or acute kidney injury.

Clause 32. The method of clause 29, wherein the first antibody and second antibody are anti-creatinine antibodies.

Clause 33. The method of clause 29, wherein the first antibody and the second antibody bind to a same epitope of the antigen.

Clause 34. The method of clause 29, wherein the first antibody and the second antibody bind to a different epitope of the antigen.

Clause 35. The method of clause 29, wherein the substrate is sucrose, the enzyme is invertase, and the product is glucose.

Clause 36. The method of clause 29, wherein the substrate is acetoacetic acid, the enzyme is βHB-hydrogenase, and the product is βhydroxybutyrate.

Clause 37. The method of clause 29, wherein the level of product is detected by a glucose meter.

Clause 38. The method of clause 37, wherein the glucose meter is integrated with a smart phone application.

Clause 39. The method of clause 29, wherein the first nanoparticle is a Fe₃O₄ nanoparticle functionalized with thiolated gold.

Clause 40. The method of clause 29, wherein the second nanoparticle is a graphene oxide nanoparticle conjugated to thiolated gold.

Clause 41. The method of clause 29, wherein the first nanoparticle is a citric acid functionalized Fe₃O₄ aqueous colloidal magnetic nanoparticle.

Clause 42. The method of clause 29, wherein the second nanoparticle is a fluorinated graphene oxide-silver nanoparticle.

Clause 43. A kit for detecting abnormal kidney function in a subject, the kit comprising:
a) a first antibody conjugated to a first nanoparticle;
b) a second antibody conjugated to a second nanoparticle, wherein the nanoparticle is conjugated to an enzyme, wherein the first and second antibodies bind an antigen; and
c) a substrate capable of being converted to a product by the enzyme.

Clause 44. The kit of clause 43, further comprising a glucose meter.

Clause 45. A method of detecting cancer in a subject, the method comprising:
a) obtaining a sample from the subject;
b) contacting the sample with:
   i) a first antibody conjugated to a first nanoparticle;
   ii) a second antibody conjugated to a second nanoparticle, wherein the nanoparticle is conjugated to an enzyme, wherein the first and second antibodies bind an antigen; and
   iii) a substrate capable of being converted to a product by the enzyme
c) determining the level of the product;
d) comparing the level of product to a reference level of product; and
e) identifying the subject as having cancer when the level of the product is different than the level of product from a reference level of product.

Clause 46. The method of clause 45 wherein the sample is selected from a tumor cell lysate, a pre-surgical biopsy, a biopsy obtained during surgery, a post-surgical biopsy, blood, plasma, urine, cerebrospinal fluid, amniotic fluid, saliva, and sweat.

Clause 47. The method of clause 45, wherein the cancer is breast cancer.

Clause 48. The method of clause 47, wherein the first antibody and the second antibody are anti-HER2 antibodies.

Clause 49. The method of clause 47, wherein the first antibody and the second antibody are anti-ER antibodies.

Clause 50. The method of any of clauses 45-49, wherein the first antibody and the second antibody bind to a same epitope of the antigen.

Clause 51. The method of any of clauses 45-49, wherein the first antibody and the second antibody bind to a different epitope of the antigen.

Clause 52. The method of clause 45, wherein the substrate is sucrose, the enzyme is invertase, and the product is glucose.

Clause 53. The method of clause 45, wherein the substrate is acetoacetic acid, the enzyme is βHB-hydrogenase, and the product is βhydroxybutyrate.

Clause 54. The method of clause 45, wherein the level of product is detected by a glucose meter.

Clause 55. The method of clause 54, wherein the glucose meter is integrated with a smart phone application.

Clause 56. The method of clause 45, wherein the first nanoparticle is a Fe₃O₄ nanoparticle functionalized with thiolated gold.

Clause 57. The method of clause 45, wherein the second nanoparticle is a graphene oxide nanoparticle conjugated to thiolated gold.

Clause 58. The method of clause 45, wherein the first nanoparticle is a citric acid functionalized Fe₃O₄ aqueous colloidal magnetic nanoparticle.

Clause 59. The method of clause 45, wherein the second nanoparticle is a fluorinated graphene oxide-silver nanoparticle.

Clause 60. A kit for detecting cancer in a subject, the kit comprising:
a) a first antibody conjugated to a first nanoparticle;
b) a second antibody conjugated to a second nanoparticle, wherein the nanoparticle is conjugated to an enzyme, wherein the first and second antibodies bind an antigen; and
c) a substrate capable of being converted to a product by the enzyme.

Clause 61. The kit of clause 63, further comprising a glucose meter.

Clause 62. A method of detecting an antigen in a subject; the method comprising:
a) obtaining a sample from a subject;
b) contacting a sample from the subject with:
   i) an antigen specific antibody conjugated to a nanoparticle;
   ii) an antigen conjugated to an enzyme; and
   iii) a substrate capable of being converted to a product by the enzyme;
c) determining the level of product; and
d) comparing the level of product to a reference level of product; thereby detecting the level of biomarker.

Clause 63. The method of clause 62, wherein the sample is selected from blood, serum, urine, cerebrospinal fluid, amniotic fluid, saliva, and sweat.

Clause 64. The method of clause 62, wherein the antigen is a biomarker.

Clause 65. The method of clause 65, wherein the biomarker is a kidney function biomarker.

Clause 66. The method of clause 65, wherein the kidney function biomarker is creatinine.

Clause 67. The method of clause 62, wherein the substrate is sucrose, the enzyme is invertase, and the product is glucose.

Clause 68. The method of clause 62, wherein the substrate is acetoacetic acid, the enzyme is βHB-hydrogenase, and the product is βhydroxybutyrate.

Clause 69. The method of clause 62, wherein the level of product is detected by a glucose meter.

Clause 70. The method of clause 69, wherein the glucose meter is integrated with a smart phone application.

Clause 71. The method of clause 62, wherein the nanoparticle is a Fe₃O₄ nanoparticle functionalized with thiolated gold.

Clause 72. The method of clause 62, wherein the nanoparticle is a citric acid functionalized Fe₃O₄ aqueous colloidal magnetic nanoparticle.

Clause 73. A kit for detecting an antigen in a sample, the kit comprising:
a) an antigen specific antibody conjugated to a nanoparticle;
b) an antigen conjugated to an enzyme; and
c) substrate capable of being converted to a product by the enzyme.

Clause 74. The kit of clause 73, further comprising a glucose meter.

Clause 75. The kit of clause 73, wherein the antigen is a biomarker.

Clause 76. The kit of clause 75, wherein the biomarker is a kidney function biomarker.

Clause 77. The kit of clause 76, wherein the kidney function biomarker is creatinine.

Clause 78.The kit of clause 73, wherein the substrate is sucrose, the enzyme is invertase, and the product is glucose.

Clause 79. The kit of clause 73, wherein the substrate is acetoacetic acid, the enzyme is βHB-hydrogenase, and the product is βhydroxybutyrate.

Clause 80. The kit of clause 79, wherein the glucose meter is integrated with a smart phone application.

Clause 81. The kit of clause 73, wherein the nanoparticle is a Fe₃O₄ nanoparticle functionalized with thiolated gold.

Clause 82. The kit of clause 73, wherein the nanoparticle is a citric acid functionalized Fe₃O₄ aqueous colloidal magnetic nanoparticle.

Clause 83. A method of detecting abnormal kidney function in a subject, the method comprising:
a) obtaining a sample from the subject;
b) contacting the sample with:
   i) an antigen specific antibody conjugated to a nanoparticle;
   ii) an antigen conjugated to an enzyme; and
   iii) a substrate capable of being converted to a product by the enzyme;
c) determining the level of product;
d) comparing the level of product to a reference level of product; and
e) identifying the subject as having abnormal kidney function when the level of product is different than the level of product from a reference level of product.

Clause 84. The method of clause 83, wherein the sample is selected from blood, serum, urine, sweat, and saliva.

Clause 85. The method of clause 83, wherein the abnormal kidney function indicates the subject has kidney impairment, kidney disease, kidney failure, kidney cancer, diabetic kidney disease, polycystic kidney disease, autosomal dominant polycystic kidney disease, kidney infection, kidney cysts, kidney stones, or acute kidney injury.

Clause 86. The method of clause 83, wherein the antigen is a biomarker.

Clause 87. The method of clause 83, wherein the antibody is an anti-creatinine antibody.

Clause 88. The method of clause 83, wherein the substrate is sucrose, the enzyme is invertase, and the product is glucose.

Clause 89. The method of clause 83, wherein the substrate is acetoacetic acid, the enzyme is βHB-hydrogenase, and the product is βhydroxybutyrate.

Clause 90. The method of clause 83, wherein the level of product is detected by a glucose meter.

Clause 91. The method of clause 84, wherein the glucose meter is integrated with a smart phone application.

Clause 92. The method of clause 83, wherein the nanoparticle is a Fe₃O₄ nanoparticle functionalized with thiolated gold.

Clause 93. The method of clause 83, wherein the nanoparticle is a citric acid functionalized Fe₃O₄ aqueous colloidal magnetic nanoparticle.

Clause 94. A kit for detecting abnormal kidney function in a subject, the kit comprising:
a) an antigen specific antibody conjugated to a nanoparticle;
b) an antigen conjugated to an enzyme; and
c) a substrate capable of being converted to a product by the enzyme.

Clause 95. The kit of clause 94, further comprising a glucose meter.

Clause 96. A method of detecting cancer in a subject, the method comprising:
a) obtaining a sample from the subject;
b) contacting the sample with:
   i) an antigen specific antibody conjugated to a nanoparticle;
   ii) an antigen conjugated to an enzyme; and
   iii) a substrate capable of being converted to a product by the enzyme;
c) determining the level of product;
d) comparing the level of product to a reference level of product; and
e) identifying the subject as having abnormal kidney function when the level of product is different than the level of product from a reference level of product.

Clause 97. The method of clause 96, wherein the sample is selected from a tumor cell lysate, a pre-surgical biopsy, a biopsy obtained during surgery, a post-surgical biopsy, blood, serum, urine, sweat, and saliva.

Clause 98. The method of clause 96, wherein the antigen is a biomarker.

Clause 99. The method of clause 96, wherein the cancer is breast cancer.

Clause 100. The method of clause 98, wherein the biomarker is HER2.

Clause 101. The method of clause 98, wherein the biomarker is ER.

Clause 102. The method of clause 98, wherein the biomarker is a metabolite.

Clause 103. The method of clause 102, wherein the metabolite is selecting from the group consisting of phosphocholine, isoleucine, threonine, glutamate, histidine, acetoactetate, glycerol, mannose, phenylalanine, and pyruvate.

Clause 104. The method of clause 102, wherein the substrate is sucrose, the enzyme is invertase, and the product is glucose.

Clause 105. The method of clause 102, wherein the substrate is acetoacetic acid, the enzyme is βHB-hydrogenase, and the product is βhydroxybutyrate.

Clause 106. The method of clause 102, wherein the level of product is detected by a glucose meter.

Clause 107. The method of clause 106, wherein the glucose meter is integrated with a smart phone application.

Clause 108. The method of clause 102, wherein the nanoparticle is a Fe₃O₄ nanoparticle functionalized with thiolated gold.

Clause 109. The method of clause 102, wherein the nanoparticle is a citric acid functionalized Fe₃O₄ aqueous colloidal magnetic nanoparticle.

Clause 110. A kit for detecting cancer in a subject, the kit comprising:
a) an antigen specific antibody conjugated to a nanoparticle;
b) an antigen conjugated to an enzyme; and
c) a substrate capable of being converted to a product by the enzyme.

Clause 111. The kit of clause 110, further comprising a glucose meter.

It is also to be understood that the following claims are intended to cover all of the generic and specific features of the invention herein described, and all statements of the scope of the invention that, as a matter of language, might be said to fall therebetween.

What is claimed is:

1. A method of detecting an antigen in a subject, the method comprising:
(a) contacting a sample from the subject with:
(i) a first antibody conjugated to a first nanoparticle;
(ii) a second antibody conjugated to a second nanoparticle, wherein the nanoparticle is conjugated to an enzyme, wherein the first and second antibodies bind an antigen; and
(iii) a substrate capable of being converted to a product by the enzyme;
(b) determining the level of the product;
(c) comparing the level of product to a reference level of product; thereby detecting the level of antigen.

2. The method of claim 1, wherein the first antibody and the second antibody bind to a same epitope of the antigen.

3. The method of claim 1, wherein the first antibody and the second antibody bind to a different epitope of the antigen.

4. The method of claim 1, wherein the sample is selected from blood, serum, urine, cerebrospinal fluid, amniotic fluid, saliva, sweat, a tumor cell lysate, a pre-surgical biopsy, a biopsy obtained during surgery, and a post-surgical biopsy.

5. The method of claim 1, wherein the antigen is a biomarker.

6. The method of claim 5, wherein the biomarker is a kidney function biomarker.

7. The method of claim 6, wherein the kidney function biomarker is creatinine.

8. The method of claim 1, wherein the substrate is sucrose, the enzyme is invertase, and the product is glucose.

9. The method of claim 1, wherein the substrate is acetoacetic acid, the enzyme is βHB-hydrogenase, and the product is βhydroxybutyrate.

10. The method of claim 1, wherein the level of product is detected by a glucose meter.

11. The method of claim 1, wherein the glucose meter is integrated with a smart phone application.

12. The method of claim 1, wherein the first nanoparticle is a Fe₃O₄ nanoparticle functionalized with thiolated gold.

13. The method of claim 1, wherein the first nanoparticle is a Fe₃O₄ nanoparticle functionalized with thiolated gold.

14. The method of claim 1, wherein the second nanoparticle is a graphene oxide nanoparticle conjugated to thiolated gold.

15. The method of claim 1, wherein the first nanoparticle is a citric acid functionalized Fe₃O₄ aqueous colloidal magnetic nanoparticle.

16. The method of claim 1, wherein the second nanoparticle is a fluorinated graphene oxide-silver nanoparticle.

17. A kit for detecting an antigen in a subject, the kit comprising:
(a) a first antibody conjugated to a first nanoparticle;
(b) a second antibody conjugated to a second nanoparticle, wherein the nanoparticle is conjugated to an enzyme, wherein the first and second antibodies bind an antigen; and
(c) a substrate capable of being converted to a product by the enzyme.

18. The kit of claim 17, further comprising a glucose meter.

19. The kit of claim 17, wherein the antigen is a biomarker.

20. The kit of claim 19, wherein the biomarker is a kidney function biomarker.

21. The kit of claim 20, wherein the kidney function biomarker is creatinine.

22. The kit of claim 17, wherein the substrate is sucrose, the enzyme is invertase, and the product is glucose.

23. The kit of claim 17, wherein the wherein the substrate is acetoacetic acid, the enzyme is βHB-hydrogenase, and the product is βhydroxybutyrate.

24. The kit of claim 18, wherein the glucose meter is integrated with a smart phone application.

25. The kit of claim 17, wherein the first nanoparticle is a Fe₃O₄ nanoparticle functionalized with thiolated gold.

26. The kit of claim 17, wherein the second nanoparticle is a graphene oxide nanoparticle conjugated to thiolated gold.

27. The kit of claim 17, wherein the first nanoparticle is a citric acid functionalized Fe₃O₄ aqueous colloidal magnetic nanoparticle.

28. The kit of claim 17, wherein the second nanoparticle is a fluorinated graphene oxide-silver nanoparticle.

29. A method of detecting abnormal kidney function in a subject, the method comprising:
(a) obtaining a sample from the subject;
(b) contacting the sample with:
(i) a first antibody conjugated to a first nanoparticle;
(ii) a second antibody conjugated to a second nanoparticle, wherein the nanoparticle is conjugated to an enzyme, wherein the first and second antibodies bind an antigen; and
(iii) a substrate capable of being converted to a product by the enzyme
(c) determining the level of the product;
(d) comparing the level of product to a reference level of product; and
(e) identifying the subject as having abnormal kidney function when the level of the product is different than the level of product from a reference level of product.

30. The method of claim 29, wherein the sample is selected from blood, serum, urine, sweat, and saliva.

31. The method of claim 29, wherein the abnormal kidney function indicates the subject has kidney impairment, kidney disease, kidney failure, kidney cancer, diabetic kidney disease, polycystic kidney disease, autosomal dominant polycystic kidney disease, kidney infection, kidney cysts, kidney stones, or acute kidney injury.

32. The method of claim 29, wherein the first antibody and second antibody are anti-creatinine antibodies.

33. The method of claim 29, wherein the first antibody and the second antibody bind to a same epitope of the antigen.

34. The method of claim 29, wherein the first antibody and the second antibody bind to a different epitope of the antigen.

35. The method of claim 29, wherein the substrate is sucrose, the enzyme is invertase, and the product is glucose.

36. The method of claim 29, wherein the substrate is acetoacetic acid, the enzyme is βHB-hydrogenase, and the product is βhydroxybutyrate.

37. The method of claim 29, wherein the level of product is detected by a glucose meter.

38. The method of claim 37, wherein the glucose meter is integrated with a smart phone application.

39. The method of claim 29, wherein the first nanoparticle is a Fe₃O₄ nanoparticle functionalized with thiolated gold.

40. The method of claim 29, wherein the second nanoparticle is a graphene oxide nanoparticle conjugated to thiolated gold.

41. The method of claim 29, wherein the first nanoparticle is a citric acid functionalized Fe₃O₄ aqueous colloidal magnetic nanoparticle.

42. The method of claim 29, wherein the second nanoparticle is a fluorinated graphene oxide-silver nanoparticle.

43. A kit for detecting abnormal kidney function in a subject, the kit comprising:
(a) a first antibody conjugated to a first nanoparticle;
(b) a second antibody conjugated to a second nanoparticle, wherein the nanoparticle is conjugated to an enzyme, wherein the first and second antibodies bind an antigen; and
(c) a substrate capable of being converted to a product by the enzyme.

44. The kit of claim 43, further comprising a glucose meter.

45. A method of detecting cancer in a subject, the method comprising:
(a) obtaining a sample from the subject;
(b) contacting the sample with:
(i) a first antibody conjugated to a first nanoparticle;
(ii) a second antibody conjugated to a second nanoparticle, wherein the nanoparticle is conjugated to an enzyme, wherein the first and second antibodies bind an antigen; and
(iii) a substrate capable of being converted to a product by the enzyme
(c) determining the level of the product;
(d) comparing the level of product to a reference level of product; and
(e) identifying the subject as having cancer when the level of the product is different than the level of product from a reference level of product.

46. The method of claim 45 wherein the sample is selected from a tumor cell lysate, a pre-surgical biopsy, a biopsy obtained during surgery, a post-surgical biopsy, blood, plasma, urine, cerebrospinal fluid, amniotic fluid, saliva, and sweat.

47. The method of claim 45, wherein the cancer is breast cancer.

48. The method of claim 47, wherein the first antibody and the second antibody are anti-HER2 antibodies.

49. The method of claim 47, wherein the first antibody and the second antibody are anti-ER antibodies.

50. The method of any of claims 45-49, wherein the first antibody and the second antibody bind to a same epitope of the antigen.

51. The method of any of claims 45-49, wherein the first antibody and the second antibody bind to a different epitope of the antigen.

52. The method of claim 45, wherein the substrate is sucrose, the enzyme is invertase, and the product is glucose.

53. The method of claim 45, wherein the substrate is acetoacetic acid, the enzyme is βHB-hydrogenase, and the product is βhydroxybutyrate.

54. The method of claim 45, wherein the level of product is detected by a glucose meter.

55. The method of claim 54, wherein the glucose meter is integrated with a smart phone application.

56. The method of claim 45, wherein the first nanoparticle is a Fe₃O₄ nanoparticle functionalized with thiolated gold.

57. The method of claim 45, wherein the second nanoparticle is a graphene oxide nanoparticle conjugated to thiolated gold.

58. The method of claim 45, wherein the first nanoparticle is a citric acid functionalized Fe₃O₄ aqueous colloidal magnetic nanoparticle.

59. The method of claim 45, wherein the second nanoparticle is a fluorinated graphene oxide-silver nanoparticle.

60. A kit for detecting cancer in a subject, the kit comprising:
(a) a first antibody conjugated to a first nanoparticle;
(b) a second antibody conjugated to a second nanoparticle, wherein the nanoparticle is conjugated to an enzyme, wherein the first and second antibodies bind an antigen; and
(c) a substrate capable of being converted to a product by the enzyme.

61. The kit of claim 63, further comprising a glucose meter.

62. A method of detecting an antigen in a subject; the method comprising:
(a) obtaining a sample from a subject;
(b) contacting a sample from the subject with:
(i) an antigen specific antibody conjugated to a nanoparticle;
(ii) an antigen conjugated to an enzyme; and
(iii) a substrate capable of being converted to a product by the enzyme;
(c) determining the level of product; and
(d) comparing the level of product to a reference level of product; thereby detecting the level of biomarker.

63. The method of claim 62, wherein the sample is selected from blood, serum, urine, cerebrospinal fluid, amniotic fluid, saliva, and sweat.

64. The method of claim 62, wherein the antigen is a biomarker.

65. The method of claim 65, wherein the biomarker is a kidney function biomarker.

66. The method of claim 65, wherein the kidney function biomarker is creatinine.

67. The method of claim 62, wherein the substrate is sucrose, the enzyme is invertase, and the product is glucose.

68. The method of claim 62, wherein the substrate is acetoacetic acid, the enzyme is βHB-hydrogenase, and the product is βhydroxybutyrate.

69. The method of claim 62, wherein the level of product is detected by a glucose meter.

70. The method of claim 69, wherein the glucose meter is integrated with a smart phone application.

71. The method of claim 62, wherein the nanoparticle is a Fe₃O₄ nanoparticle functionalized with thiolated gold.

72. The method of claim 62, wherein the nanoparticle is a citric acid functionalized Fe₃O₄ aqueous colloidal magnetic nanoparticle.

73. A kit for detecting an antigen in a sample, the kit comprising:
(a) an antigen specific antibody conjugated to a nanoparticle;
(b) an antigen conjugated to an enzyme; and
(c) a substrate capable of being converted to a product by the enzyme.

74. The kit of claim 73, further comprising a glucose meter.

75. The kit of claim 73, wherein the antigen is a biomarker.

76. The kit of claim 75, wherein the biomarker is a kidney function biomarker.

77. The kit of claim 76, wherein the kidney function biomarker is creatinine.

78. The kit of claim 73, wherein the substrate is sucrose, the enzyme is invertase, and the product is glucose.

79. The kit of claim 73, wherein the substrate is acetoacetic acid, the enzyme is βHB-hydrogenase, and the product is βhydroxybutyrate.

80. The kit of claim 79, wherein the glucose meter is integrated with a smart phone application.

81. The kit of claim 73, wherein the nanoparticle is a Fe₃O₄ nanoparticle functionalized with thiolated gold.

82. The kit of claim 73, wherein the nanoparticle is a citric acid functionalized Fe₃O₄ aqueous colloidal magnetic nanoparticle.

83. A method of detecting abnormal kidney function in a subject, the method comprising:
(a) obtaining a sample from the subject;
(b) contacting the sample with:
(i) an antigen specific antibody conjugated to a nanoparticle;
(ii) an antigen conjugated to an enzyme; and
(iii) a substrate capable of being converted to a product by the enzyme;
(c) determining the level of product;
(d) comparing the level of product to a reference level of product; and
(e) identifying the subject as having abnormal kidney function when the level of product is different than the level of product from a reference level of product.

84. The method of claim 83, wherein the sample is selected from blood, serum, urine, sweat, and saliva.

85. The method of claim 83, wherein the abnormal kidney function indicates the subject has kidney impairment, kidney disease, kidney failure, kidney cancer, diabetic kidney disease, polycystic kidney disease, autosomal dominant polycystic kidney disease, kidney infection, kidney cysts, kidney stones, or acute kidney injury.

86. The method of claim 83, wherein the antigen is a biomarker.

87. The method of claim 83, wherein the antibody is an anti-creatinine antibody.

88. The method of claim 83, wherein the substrate is sucrose, the enzyme is invertase, and the product is glucose.

89. The method of claim 83, wherein the substrate is acetoacetic acid, the enzyme is βHB-hydrogenase, and the product is βhydroxybutyrate.

90. The method of claim 83, wherein the level of product is detected by a glucose meter.

91. The method of claim 84, wherein the glucose meter is integrated with a smart phone application.

92. The method of claim 83, wherein the nanoparticle is a Fe₃O₄ nanoparticle functionalized with thiolated gold.

93. The method of claim 83, wherein the nanoparticle is a citric acid functionalized Fe₃O₄ aqueous colloidal magnetic nanoparticle.

94. A kit for detecting abnormal kidney function in a subject, the kit comprising:
(a) an antigen specific antibody conjugated to a nanoparticle;
(b) an antigen conjugated to an enzyme; and
(c) a substrate capable of being converted to a product by the enzyme.

95. The kit of claim 94, further comprising a glucose meter.

96. A method of detecting cancer in a subject, the method comprising:
(a) obtaining a sample from the subject;
(b) contacting the sample with:
(i) an antigen specific antibody conjugated to a nanoparticle;
(ii) an antigen conjugated to an enzyme; and
(iii) a substrate capable of being converted to a product by the enzyme;
(c) determining the level of product;
(d) comparing the level of product to a reference level of product; and
(e) identifying the subject as having abnormal kidney function when the level of product is different than the level of product from a reference level of product.

97. The method of claim 96, wherein the sample is selected from a tumor cell lysate, a pre-surgical biopsy, a biopsy obtained during surgery, a post-surgical biopsy, blood, serum, urine, sweat, and saliva.

98. The method of claim 96, wherein the antigen is a biomarker.

99. The method of claim 96, wherein the cancer is breast cancer.

100. The method of claim 98, wherein the biomarker is HER2.

101. The method of claim 98, wherein the biomarker is ER.

102. The method of claim 98, wherein the biomarker is a metabolite.

103. The method of claim 102, wherein the metabolite is selecting from the group consisting of phosphocholine, isoleucine, threonine, glutamate, histidine, acetoactetate, glycerol, mannose, phenylalanine, and pyruvate.

104. The method of claim 102, wherein the substrate is sucrose, the enzyme is invertase, and the product is glucose.

105. The method of claim 102, wherein the substrate is acetoacetic acid, the enzyme is βHB-hydrogenase, and the product is βhydroxybutyrate.

106. The method of claim 102, wherein the level of product is detected by a glucose meter.

107. The method of claim 106, wherein the glucose meter is integrated with a smart phone application.

108. The method of claim 102, wherein the nanoparticle is a Fe₃O₄ nanoparticle functionalized with thiolated gold.

109. The method of claim 102, wherein the nanoparticle is a citric acid functionalized Fe₃O₄ aqueous colloidal magnetic nanoparticle.

110. A kit for detecting cancer in a subject, the kit comprising:
(a) an antigen specific antibody conjugated to a nanoparticle;
(b) an antigen conjugated to an enzyme; and
(c) a substrate capable of being converted to a product by the enzyme.

111. The kit of claim 110, further comprising a glucose meter.
